(19) United States
(12) Patent Application Publication
(10) Pub. No.: US 2023/0154611 A1
(43) Pub. Date: May 18, 2023

(54) METHODS AND SYSTEMS FOR DETECTING STROKE IN A PATIENT

(71) Applicant: V GROUP INC., Cranbury, NJ (US)

(72) Inventors: Vijay Kumar PALANDURKAR, West Windsor Township, NJ (US); Aakash Haresh SAVITA, West Lafayette, IN (US)

(21) Appl. No.: 17/530,045

(22) Filed: Nov. 18, 2021

(51) Int. Cl. G16H 50/20; G16H 10/60; G16H 40/67; G06F 1/16; G06K 9/00; G06K 9/62; G06T 7/00; G10L 15/02; A61B 5/00; A61B 5/11

(52) U.S. Cl. CPC G16H 50/20; G16H 10/60; G16H 40/67; G06F 1/163; G06K 9/00228; G06K 9/6256; G06K 9/6261; G06T 7/0014; G10L 15/02; A61B 5/4803; A61B 5/1128; A61B 5/0051; A61B 5/4824; A61B 5/747; A61B 5/7264; A61B 5/7267; G06K 2209/05; G06T 2207/10016; G06T 2207/30201; G06T 2207/20081

(57) ABSTRACT

Embodiments of the present disclosure provide systems and methods for performing stroke detection with machine learning (ML) systems. The method performed by a computer system includes accessing a video of a user. The method includes performing a first test on the accessed video for detecting a facial drooping factor and speech slur factor of the user in real-time. The facial drooping factor is detected with facilitation of one or more techniques. The speech slur factor is detected with execution of machine learning algorithms. The method includes performing a second test on the user for detecting a numbness factor in hands of the user. The method includes processing the facial drooping factor, the speech slur factor, and the numbness factor for detecting symptoms of stroke in the user in real-time. The method includes sending notification to at least one emergency contact of the user in real-time for providing medical assistance.

### TECHNICAL FIELD

The present disclosure relates to machine learning models and, more particularly relates, to systems and methods for detecting the occurrence or prediction of stroke in a patient.

### BACKGROUND

A cerebrovascular accident, commonly known as ‘stroke’, is a medical condition that arises due to a lack of oxygen to the brain. Stoke may cause permanent brain damage or death if not treated on time. The stroke may be an ischemic stroke or hemorrhagic stroke. Generally, ischemic stroke occurs because of a blocked artery and hemorrhagic stroke occurs due to leaking or bursting of a blood vessel. Ischemic strokes may be further classified as thrombotic strokes and embolic strokes. Hemorrhagic strokes may be further classified as intracerebral strokes and subarachnoid strokes. The strokes result in a decrease in the amount of oxygen supplied to the brain, which may further cause the brain cells to become damaged. Symptoms of a stroke may include trouble in speaking and understanding, paralysis/numbness in the face, arm, or leg, trouble seeing, headache, trouble walking, and so on.

In case of a stroke, providing emergency treatment to the patient is important to reduce the chance of permanent disability or death. Currently, various steps are taken to diagnose stroke in the patient and treatment for the stroke. Initially, a physical examination is required by a medical practitioner to rule out the possibility of other health issues such as brain tumors or reactions due to drugs. After the physical examination, blood samples of the patient might be taken to determine how fast the patient's blood clots and to check chemical balances and blood sugar levels.

Further, the patient may need to undergo CT scans and MRI scans. Generally, a CT scan (computerized tomography scan) is performed by injecting dye into the patient and viewing the brain to determine whether the issue is a stroke or a different health problem. Additionally, MRI (Magnetic Resonance Imaging) allows the medical practitioner to look at the brain of the patient to see damaged tissues caused by the potential stroke. Additionally, an echocardiogram might be performed to find out if and where the blood clots are occurring in the heart. However, current methods of performing stroke detection are manual, time-consuming, and costly because they require the use of heavy and expensive equipment. In addition, government regulations and the approval process of new drugs and devices cause a hindrance in providing treatment to the patient.

Therefore, there is a need for techniques to overcome one or more limitations stated above in addition to providing other technical advantages.

### SUMMARY

Various embodiments of the present disclosure provide systems and methods for performing the detection of stroke with machine learning (ML) systems.

In an embodiment, a computer-implemented method is disclosed. The computer-implemented method performed by a computer system includes accessing a video of a user in real-time. The video of the user is recorded for a first interval of time. The method includes performing a first test on the accessed video for detecting a facial drooping factor and a speech slur factor of the user in real-time. The facial drooping factor is detected with the facilitation of one or more techniques. The speech slur factor is detected with the execution of machine learning algorithms. The method includes performing a second test on the user for a second interval of time. The second test is a vibration test performed for detecting a numbness factor in hands of the user. The method includes processing the facial drooping factor, the speech slur factor, and the numbness factor for detecting symptoms of stroke in the user in real-time. The method includes sending notifications to at least one emergency contact of the user in real-time for providing medical assistance to the user. The notification is sent upon detection of symptoms of stroke in the user.

In another embodiment, a computer system is disclosed. The computer system includes one or more sensors. The computer system includes a memory including executable instructions and a processor. The processor is configured to execute the instructions to cause the computer system to at least access a video of a user in real-time. The video of the user is recorded for a first interval of time. The computer system is caused to perform a first test on the accessed video to detect a facial drooping factor and a speech slur factor of the user in real-time. The facial drooping factor is detected with the facilitation of one or more techniques. The speech slur factor is detected with the execution of machine learning algorithms. The computer system is caused to perform a second test on the user for a second interval of time. The second test is a vibration test performed to detect a numbness factor in hands of the user. The computer system is caused to process the facial drooping factor, the speech slur factor, and the numbness factor to detect symptoms of stroke in the user in real-time. The computer system is caused to send a notification to at least one emergency contact of the user in real-time to provide medical assistance to the user. The notification is sent upon detection of symptoms of stroke in the user.

In yet another embodiment, a server system is disclosed. The server system includes a communication interface. The server system includes a memory including executable instructions and a processing system communicably coupled to the communication interface. The processor is configured to execute the instructions to cause the server system to provide an application to a computer system. The computer system includes one or more sensors, a memory to store the application in a machine-executable form, and a processor. The application is executed by the processor in the computer system to cause the computer system to perform a method. The method performed by the computer system includes accessing a video of a user in real-time. The video of the user is recorded for a first interval of time. The method includes performing a first test on the accessed video for detecting a facial drooping factor and a speech slur factor of the user in real-time. The facial drooping factor is detected with the facilitation of one or more techniques. The speech slur factor is detected with the execution of machine learning algorithms. The method includes performing a second test on the user for a second interval of time. The second test is a vibration test performed for detecting a numbness factor in hands of the user. The method includes processing the facial drooping factor, the speech slur factor, and the numbness factor for detecting symptoms of stroke in the user in real-time. The method includes sending notifications to at least one emergency contact of the user in real-time for providing medical assistance to the user. The notification is sent upon detection of symptoms of stroke in the user.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device or a tool and instrumentalities disclosed herein. Moreover, those in the art will understand that the drawings are not to scale. Wherever possible, like elements have been indicated by identical numbers:

FIG. 1 is an illustration of an environment related to at least some example embodiments of the present disclosure;

FIG. 2 is a simplified block diagram of a server system, in accordance with one embodiment of the present disclosure;

FIG. 3 is a data flow diagram representation for performing stroke detection in real-time, in accordance with an embodiment of the present disclosure;

FIG. 4 is a simplified data flow diagram representation for performing stroke detection using a first technique of one or more techniques, in accordance with an embodiment of the present disclosure;

FIG. 5 is a simplified data flow diagram representation for performing stroke detection using a second technique of the one or more techniques, in accordance with an embodiment of the present disclosure;

FIG. 6A is a high-level data flow diagram representation for performing stroke detection using the first technique and the second technique of the one or more techniques, in accordance with an example embodiment of the present disclosure;

FIG. 6B is a high-level data flow diagram representation for performing stroke detection using a third technique of the one or more techniques, in accordance with an embodiment of the present disclosure;

FIG. 7A is a schematic representation of a process for training a deep learning model for detecting facial drooping factor, in accordance with an embodiment of the present disclosure;

FIG. 7B is a schematic representation of a process for implementation of the deep learning model for detecting facial drooping factor in real-time, in accordance with an embodiment of the present disclosure;

FIG. 8 is a simplified data flow diagram representation for detecting speech slur factor in voice of the user in real-time, in accordance with an embodiment of the present disclosure;

FIG. 9 is a simplified data flow diagram representation for detecting numbness factor in hands of the user in real-time, in accordance with an embodiment of the present disclosure;

FIGS. 10A-10C, collectively, represent user interfaces (UIs) of application for setting up an emergency contact to notify in case symptoms of a stroke are detected in the user, in accordance with an embodiment of the present disclosure;

FIGS. 11A-11C, collectively, represent UIs of application for performing a first test for performing stroke detection, in accordance with an embodiment of the present disclosure;

FIGS. 12A-12C, collectively, represent UIs of application for performing a second test for stroke detection, in accordance with an embodiment of the present disclosure;

FIGS. 13A-13C, collectively, represent user interfaces (UIs) of application for processing results of the first test and the second test for performing stroke detection, in accordance with an embodiment of the present disclosure;

FIG. 14 is a process flow chart of a computer-implemented method for performing stroke detection, in accordance with an embodiment of the present disclosure; and

FIG. 15 is a simplified block diagram of an electronic device capable of implementing various embodiments of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

Various embodiments of the present disclosure provide methods and systems for detecting stroke in a patient in real-time. The system performs various tests to detect symptoms of stroke in the patient. In one embodiment, the stroke is ischemic stroke. In another embodiment, the stroke may be hemorrhagic stroke.

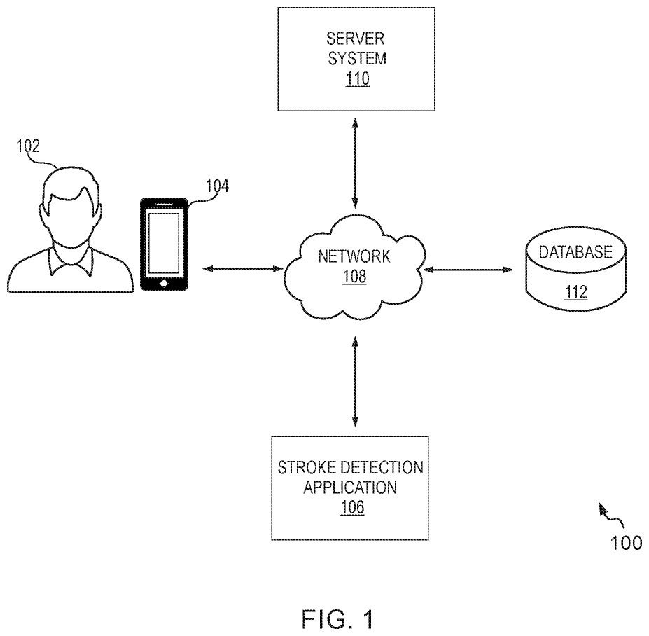

Various example embodiments of the present disclosure are described hereinafter with reference to FIGS. 1 to 15.

FIG. 1 illustrates an exemplary representation of an environment 100 related to at least some example embodiments. Although the environment 100 is presented in one arrangement, other embodiments may include the parts of the environment 100 (or other parts) arranged otherwise depending on, for example, sending notifications from various systems, performing a first test and a second test on a user 102 and processing results of the first test and the second test for detecting symptoms of stroke in the user 102. The environment 100 generally includes the user 102, a user device 104, a server system 110, a database 112, and a stroke detection application 106, each coupled to, and in communication with (and/or with access to) a network 108. The network 108 may include, without limitation, a light fidelity (Li-Fi) network, a local area network (LAN), a wide area network (WAN), a metropolitan area network (MAN), a satellite network, the Internet, a fiber optic network, a coaxial cable network, an infrared (IR) network, a radio frequency (RF) network, a virtual network, and/or another suitable public and/or private network capable of supporting communication among the entities illustrated in FIG. 1, or any combination thereof.

Various entities in the environment 100 may connect to the network 108 in accordance with various wired and wireless communication protocols, such as, Transmission Control Protocol and Internet Protocol (TCP/IP), User Datagram Protocol (UDP), 2nd Generation (2G), 3rd Generation (3G), 4th Generation (4G), 5th Generation (5G) communication protocols, Long Term Evolution (LTE) communication protocols, any future communication protocol, or any combination thereof. In some instances, the network 108 may include a secure protocol (e.g., Hypertext Transfer Protocol (HTTP)), and/or any other protocol, or set of protocols. In an example embodiment, the network 108 may include, without limitation, a local area network (LAN), a wide area network (WAN) (e.g., the Internet), a mobile network, a virtual network, and/or another suitable public and/or private network capable of supporting communication among two or more of the entities illustrated in FIG. 1, or any combination thereof.

The user 102 is a person that operates the user device 104 in real-time to detect symptoms of stroke. The user 102 may launch the stroke detection application 106 installed in the user device 104. The user device 104 is associated with the user 102. Examples of the user device 104 may include, without limitation, smart phones, tablet computers, other handheld computers, wearable devices, laptop computers, desktop computers, servers, portable media players, gaming devices, PDAs and so forth. In an embodiment, the user device 104 may host, manage, or execute the stroke detection application 106 that can interact with the database 112. In another embodiment, the user device 104 may be equipped with an instance of the stroke detection application 106.

In one embodiment, the user device 104 may include one or more sensors. The one or more sensors may include, at least one of, a motion detector, an accelerometer, a gyroscope, a microphone, a camera, a temperature sensor, an ECG sensor, and the like.

In an embodiment, the stroke detection application 106 may be or include a web browser which the user 102 may use to navigate to a website used to perform stroke detection. As another example, the stroke detection application 106 may include a mobile application or “app”. For example, the stroke detection application 106 is a mobile application installed in an Android-based smartphone, or an iOS-based iPhone or iPad operated by the user 102 to perform stroke detection in real-time. In another example, the stroke detection application 106 may include background processes that perform various operations without direct interaction from the user 102. The stroke detection application 106 may include a “plug-in” or “extension” to another application, such as a web browser plug-in or extension.

In one embodiment, the stroke detection application 106 is installed in the user device 104 associated with the user 102. In another embodiment, the stroke detection application 106 is managed, hosted, or executed by the server system 110. In yet another embodiment, the server system 110 provides the stroke detection application 106. The stroke detection application 106 is configured to display various graphical user interfaces (GUIs) to the user 102 for detecting symptoms of stroke in the user 102 in real-time.

The user 102 launches the stroke detection application 106 on the user device 104. The stroke detection application 106 notifies the user 102 to record a video of face of the user 102 in real-time. The stroke detection application 106 further accesses the video of the user 102 in real-time. The stroke detection application 106 records the video of the user 102 for a first interval of time. In one non-limiting example, the first interval of time is 5 seconds. However, the first interval of time can be any other suitable value also such as 10 seconds, 20 seconds or any other value.

The stroke detection application 106 performs the first test on the accessed video to detect a facial drooping factor and a speech slur factor of the user 102 in real-time. In addition, the stroke detection application 106 detects the facial drooping factor with the facilitation of one or more techniques. Further, the stroke detection application 106 detects the speech slur factor with the execution of machine learning algorithms. In an embodiment, these machine learning algorithms are mobile application-run machine learning algorithms.

The one or more techniques include a first technique of utilization of a machine learning model to scan the entire face of the user 102 recorded in the accessed video to detect the facial drooping factor in face of the user 102. The one or more techniques further include a second technique of utilization of a deep learning model to segment the face of the user 102 recorded in the accessed video into a plurality of facial segments in real-time. The deep learning model scans each of the plurality of facial segments to detect the facial drooping factor in face of the user 102.

In one example, the plurality of facial segments includes right-left eyes, right-left eyebrows, lips, cheeks, jaw line, and the like.

The one or more techniques also include a third technique to compare the face of the user 102 recorded in the accessed video in real-time with the face of the user 102 already stored in the database 112. In an embodiment, the comparison is performed by the stroke detection application 106. The stroke detection application 106 uses the third technique of the one or more techniques to detect stroke in the user 102. For example, the stroke detection application 106 finds the difference between the face of the user 102 recorded in the accessed video with the face of the user 102 already stored in the database 112. The stroke detection application 106 performs the comparison to detect the facial drooping factor in face of the user 102 recorded in the accessed video in real-time.

In an embodiment, the stroke detection application 106 is installed in a wearable device. In another embodiment, a third-party application (i.e., related to health and fitness) is installed in the wearable device. The wearable device is worn by the user 102. The wearable device transmits additional health information of the user 102 to the user device 104 in real-time. For example, a health application installed inside the wearable device (e.g., a smart watch) synchronizes with the stroke detection application 106 to transmit additional health information of the user 102 such as activity, body measurements, cycle tracking (if applicable), heart rate, nutrition, respiratory, sleep pattern, symptoms, body vital, and the like.

In one embodiment, the stroke detection application 106 may use any of the one or more techniques to detect the facial drooping factor in the user 102 in real-time. The stroke detection application 106 detects the speech slur factor with the facilitation of the machine learning model capapble of being executed by processing capabilities of a smartphone having mobile applications

The stroke detection application 106 performs the second test on the user 102 for a second interval of time. In one example, the second interval of time is 7 seconds. In another example, the second interval of time is 14 seconds. In yet another example, the second interval of time is of any other time. The second test is a vibration test performed by the stroke detection application 106 to detect a numbness factor in hands of the user 102.

The stroke detection application 106 processes the facial drooping factor, the speech slur factor, and the numbness factor for detecting symptoms of stroke in the user 102 in real-time. In one example, the stroke detection application 106 compares the facial drooping factor with a threshold value to detect whether there is facial drooping in the user 102 or not. In another example, the stroke detection application 106 compares the speech slur factor with a threshold value to detect whether there is a speech slur in the user 102 or not. In another example, the stroke detection application 106 detects the numbness factor by asking the user 102 if the user 102 feels the vibration of the user device 104 while holding the user device 104 in hands. Based on the response from the user 102, the stroke detection application 106 detects the numbness factor in the hands of the user 102.

The stroke detection application 106 detects the symptoms of stroke in the user 102 based on the processing of the facial drooping factor, the speech slur factor, and the numbness factor. The stroke detection application 106 further sends a notification to at least one emergency contact of the user 102 in real-time to provide medical assistance to the user 102. The notification is sent only upon detection of symptoms of stroke in the user 102. In one embodiment, the notification may include a text, SMS, call, geo-location coordinates of the user 102, and the like.

In an example, user A is undergoing stroke attack in real-time. When the user A is undergoing the stroke attack, facial features of the user A such as eyebrows, nose, lips and so on will not remain at the same level and will get distorted. The stroke detection application 106 considers this distortion of the facial features of the user A to detect the facial drooping factor of the user A.

Similarly, the stroke detection application 106 performs speech analysis of voice of the user A to detect the speech slur factor of the user A. The stroke detection application 106 identifies speech anomalies in the voice of the user A to detect the speech slur factor of the user A.

In addition, the server system 110 should be understood to be embodied in at least one computing device in communication with the network 108, which may be specifically configured, via executable instructions, to perform as described herein, and/or to be embodied in at least one non-transitory computer-readable media. In one embodiment, the stroke detection application 106 is an application/tool resting at the server system 110.

In an embodiment, the server system 110 may implement the backend APIs corresponding to the stroke detection application 106 which instructs the server system 110 to perform one or more operations described herein. In one example, the server system 110 is configured to invoke the stroke detection application 106 installed in the user device 104. In addition, the server system 110 is configured to access video of the user 102 being recorded in the user device 104 in real-time. The server system 110 is further configured to perform the first test on the accessed video of the user 102 for detecting the facial drooping factor and the speech slur factor of the user 102.

Furthermore, the server system 110 may be configured to perform the second test on the user 102 for a second interval of time. More specifically, the server system 110 performs the vibration test on the user 102 for detecting the numbness factor in hands of the user 102. The server system 110 processes the facial drooping factor, the speech slur factor, and the numbness factor for detecting symptoms of stroke in the user 102. The server system 110 also sends notifications to at least one emergency contact of the user 102 for providing medical assistance to the user 102. The server system 110 should be understood to be embodied in at least one computing device in communication with the network 108, which may be specifically configured, via executable instructions, to perform as described herein, and/or embodied in at least one non-transitory computer-readable media.

In an embodiment, the server system 110 may include one or more databases, such as the database 112. The database 112 may be configured to store a user profile of the user 102. The user profile includes data such as, but not limited to, demographic information of the user 102, images and videos of the user 102, voice samples and speech data of the user 102, and health information (e.g., heart rate information, blood oxygen level information etc.) of the user 102. The user profile is stored for personalized health reporting of the user 102.

The number and arrangement of systems, devices, and/or networks shown in FIG. 1 are provided as an example. There may be additional systems, devices, and/or networks; fewer systems, devices, and/or networks; different systems, devices, and/or networks, and/or differently arranged systems, devices, and/or networks than those shown in FIG. 1. Furthermore, two or more systems or devices shown in FIG. 1 may be implemented within a single system or device, or a single system or device shown in FIG. 1 may be implemented as multiple, distributed systems or devices. Additionally, or alternatively, a set of systems (e.g., one or more systems) or a set of devices (e.g., one or more devices) of the environment 100 may perform one or more functions described as being performed by another set of systems or another set of devices of the environment 100.

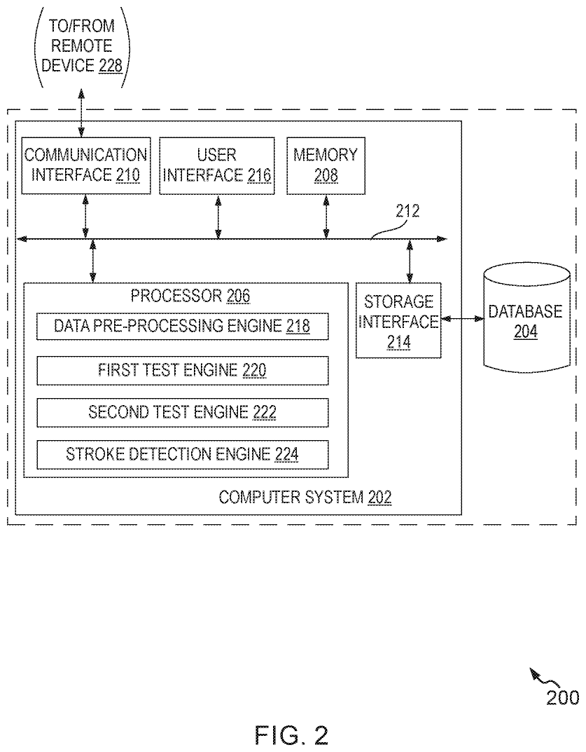

FIG. 2 is a simplified block diagram of a server system 200, in accordance with one embodiment of the present disclosure. Examples of the server system 200 include, but are not limited to, the server system 110 as shown in FIG. 1. In some embodiments, the server system 200 is embodied as a cloud-based and/or SaaS-based (software as a service) architecture.

The server system 200 includes a computer system 202 and a database 204. The computer system 202 includes at least one processor 206 for executing instructions, a memory 208, a communication interface 210, a storage interface 214, and a user interface 216. The one or more components of the computer system 202 communicate with each other via a bus 212. The components of the server system 200 provided herein may not be exhaustive and that the server system 200 may include more or fewer components than those depicted in FIG. 2. Further, two or more components may be embodied in one single component, and/or one component may be configured using multiple sub-components to achieve the desired functionalities.

In one embodiment, the database 204 is integrated within the computer system 202 and configured to store an instance of the stroke detection application 106 and one or more components of the stroke detection application 106. The one or more components of the stroke detection application 106 may be, but are not limited to, information related to warnings or notifications, settings for setting up emergency contacts for sending the notifications, and the like. The computer system 202 may include one or more hard disk drives as the database 204. The storage interface 214 is any component capable of providing the processor 206 an access to the database 204. The storage interface 214 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing the processor 206 with access to the database 204.

The processor 206 includes suitable logic, circuitry, and/or interfaces to execute computer-readable instructions for performing stroke detection in real-time. Examples of the processor 206 include, but are not limited to, an application-specific integrated circuit (ASIC) processor, a reduced instruction set computing (RISC) processor, a complex instruction set computing (CISC) processor, a field-programmable gate array (FPGA), and the like. The memory 208 includes suitable logic, circuitry, and/or interfaces to store a set of computer-readable instructions for performing operations. Examples of the memory 208 include a random-access memory (RAM), a read-only memory (ROM), a removable storage drive, a hard disk drive (HDD), and the like. It will be apparent to a person skilled in the art that the scope of the disclosure is not limited to realizing the memory 208 in the server system 200, as described herein. In some embodiments, the memory 208 may be realized in the form of a database server or a cloud storage working in conjunction with the server system 200, without deviating from the scope of the present disclosure. In some embodiments, the memory 208 may be realized in the form of a database server or a cloud storage working in conjunction with the server system 200, without deviating from the scope of the present disclosure.

The processor 206 is operatively coupled to the communication interface 210 such that the processor 206 is capable of communicating with a remote device 228 such as, the user device 104, or with any entity connected to the network 108 (e.g., as shown in FIG. 1). In one embodiment, the processor 206 is configured to invoke the stroke detection application 106 that further performs the first test and the second test for detecting symptoms of stroke in the user 102 in real-time.

It is noted that the server system 200 as illustrated and hereinafter described is merely illustrative of an apparatus that could benefit from embodiments of the present disclosure and, therefore, should not be taken to limit the scope of the present disclosure. It is noted that the server system 200 may include fewer or more components than those depicted in FIG. 2.

In one embodiment, the processor 206 includes a training engine 218, a first test engine 220, a second test engine 222 and a stroke detection engine 224. It should be noted that the components, described herein, can be configured in a variety of ways, including electronic circuitries, digital arithmetic and logic blocks, and memory systems in combination with software, firmware, and embedded technologies.

In one embodiment, the training engine 218 includes a suitable logic and/or interfaces for training the machine learning model to perform the first test, the result of which further leads to stroke detection in real-time. The training engine 218 receives sample facial data sets of non-facial muscle drooped images (normal images) and facial muscle drooped images (disease state images) of one or more users. The training engine 218 further trains the machine learning model with the sample facial data sets to scan the entire face of the user 102 recorded in the accessed video to detect the facial drooping factor in the user 102 in real-time. The training engine 218 utilizes the first technique of the one or more techniques to train the machine learning model to detect the facial drooping factor in the entire face of the user 102.

In another embodiment, the training engine 218 includes a suitable logic and/or interfaces for training the deep learning model (or a plurality of machine learning models) to perform the first test, the result of which further leads to stroke detection in real-time. The training engine 218 receives sample facial data sets of non-facial muscle drooped images (normal images) and facial muscle drooped images (disease state images) of one or more users. The training engine 218 further segments the face of the user 102 recorded in the accessed video in the plurality of facial segments in real-time. In one example, the plurality of facial segments includes right-left eyes, right-left eyebrows, lips, cheeks, jaw line, and the like. Furthermore, the training engine 218 is trained based on the sample facial data sets to detect the facial drooping factor in the face of the user 102 in real-time. The training engine 218 utilizes the second technique of the one or more techniques to train the deep learning model to detect the facial drooping factor by accessing the plurality of facial segments of the user 102.

In yet another embodiment, the training engine 218 receives image samples of the face of the user 102 at an initial step as part of the calibration process. In addition, the training engine 218 receives voice samples (audio samples) of the user 102 at an initial step as part of the calibration process. Further, the training engine 218 trains the machine learning model with sample speech data sets of non-audio slur audio and audio slur audio of one or more users.

The training engine 218 trains the machine learning model and the deep learning model using a convolutional neural network model. In general, a convolutional neural network is a deep learning algorithm mainly used for problems such as image classification. In addition, a convolutional neural network receives an image as input, assigns learnable weight and biases to various segments in the image, to be able to differentiate the various segments from the other. The training engine 218 trains the machine learning model to perform stroke detection based on detection of the facial drooping factor of the user 102 in real-time. The training engine 218 also trains the deep learning model to perform stroke detection based on detection of the facial drooping factor of the user 102 in real-time.

The training engine 218 is also trained to detect the speech slur in the voice of the user 102 in real-time. The training engine 218 receives sample speech data sets of both non-audio slur (normal state) and audio slur (disease state). Further, the training engine 218 is trained on the sample speech data sets using the machine learning models.

The first test engine 220 includes a suitable logic and/or interfaces for performing the first test in real-time for detecting the facial drooping factor and the speech slur factor in the user 102. In one embodiment, the first test engine 220 utilizes the first technique of the one or more techniques to detect the facial drooping factor of the user 102. In another embodiment, the first test engine 220 utilizes the second technique of the one or more techniques to detect the facial drooping factor of the user 102. In yet another embodiment, the first test engine 220 performs a comparison between the real-time face of the user 102 recorded in the accessed video with the face of the user 102 already stored in the database 112 at the initial step as part of the calibration process, to detect the facial drooping of the user 102.

In addition, the first test engine 220 utilizes the machine learning models to detect the speech slur factor in the recorded video of the user 102 in the user device 104. In one example, the first test engine 220 extracts audio from the recorded video of the user 102. The first test engine 220 further detects whether the recorded audio has the speech slur or not, with the execution of the machine learning models. In one embodiment, the first test engine 220 detects the speech slur factor by comparing the real-time audio of the user 102 recorded in the accessed video with the audio of the user 102 stored in the database 112. In one example, the first test engine 220 compares factors that may include, but may not be limited to, modulation of speech, high notes, low notes, and time taken by the user 102 to speak the specific phrase to detect the speech slur factor of the user 102. In one example, based on the analysis of the facial drooping factor and the speech slur factor, results of the first test are computed by the first test engine 220.

The second test engine 222 includes a suitable logic and/or interfaces for performing the second test on the user 102 with facilitation of the user device 104. The second test engine 222 performs the second test for the second interval of time. In an example, the second interval of time is of 10 seconds. In another example, the second interval of time is of 15 seconds. In yet another example, the second time interval is of 20 seconds. In yet another example, the second time interval is of any other time.

The second test engine 222 performs the second test to detect the numbness factor in hands of the user 102. The second test is the vibration test performed to detect the steadiness of the hands of the user 102 in real-time while holding the user device 104. In one example, based on analysis of the numbness factor, results of the second test are computed by the second test engine 222.

The stroke detection engine 224 includes a suitable logic and/or interfaces for processing the facial drooping factor, the speech slur factor, and the numbness factor for detecting symptoms of stroke in the user 102. The stroke detection engine 224 detects the symptoms of stroke in the user 102 in real-time. The stroke detection engine 224 further sends a notification to at least one emergency contact of the user 102 in real-time if symptoms of stroke are detected in the user 102. The stroke detection engine 224 sends a notification to the emergency contact to provide medical assistance to the user 102. The user 102 may set any number of contacts as emergency contacts. If the symptoms of stroke are not detected in the user 102, the stroke detection engine 224 informs the user 102 that stroke is not detected in the user 102.

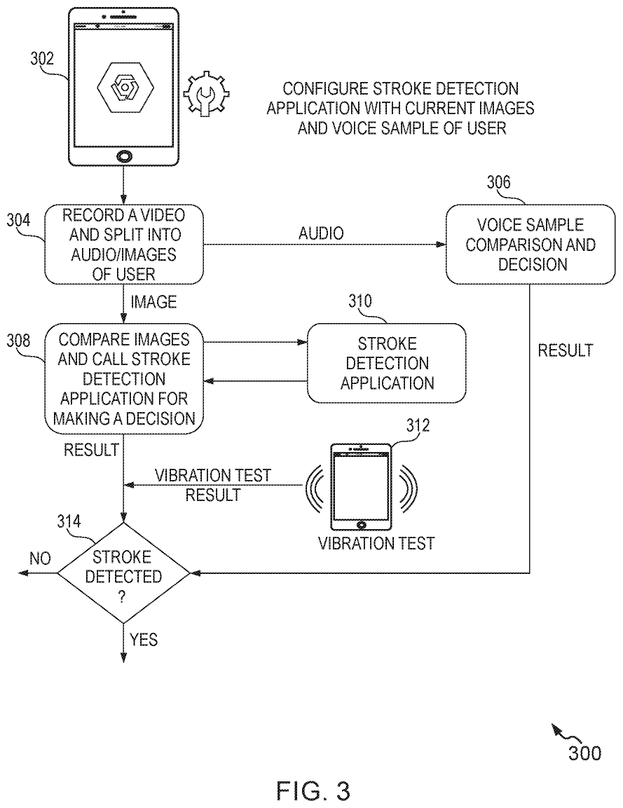

FIG. 3 is a data flow diagram representation 300 for performing stroke detection in real-time, in accordance with an embodiment of the present disclosure. It should be appreciated that each operation explained in the representation 300 is performed by the stroke detection application 106. The sequence of operations of the representation 300 may not be necessarily executed in the same order as they are presented. Further, one or more operations may be grouped together and performed in form of a single step, or one operation may have several sub-steps that may be performed in parallel or in a sequential manner. It is to be noted that to explain the process steps of FIG. 3, references may be made to system elements of FIG. 1 and FIG. 2.

At 302, the stroke detection application 106 is configured with images and voice samples of the user 102. The stroke detection application 106 is calibrated with video (for image and voice samples) of the user 102 as an initial step. The stroke detection application 106 stores video of the user 102 in the database 112. The stroke detection application 106 displays instructions to the user 102 to speak a specific phrase in the user device 104 to collect voice samples of the user 102.

At 304, the stroke detection application 106 displays instructions to the user 102 to record a video in the user device 104. In addition, the stroke detection application 106 splits the video into audio samples and images of face of the user 102 in real-time.

At 306, the stroke detection application 106 performs a comparison of the recorded voice samples with the voice samples of the user 102 already stored in the database 112 to detect the speech slur factor of the user 102 in real-time.

At 308, the stroke detection application 106 performs a comparison between the recorded images of the user 102 in the real-time video and the images of the user 102 already stored in the database 112 as part of an initial step.

At 310, the stroke detection application 106 detects the facial drooping factor of the user 102 in real-time. The first test includes the facial drooping test as well as the speech slur test. The stroke detection application 106 provides result of the first test in form of the facial drooping factor and the speech slur factor.

At 312, the stroke detection application 106 performs the second test. The second test is the vibration test that is performed on the user device 104 to detect the numbness factor in hands of the user 102.

At 314, the stroke detection application 106 processes the facial drooping factor, the speech slur factor, and the numbness factor to detect symptoms of stroke present in the user 102. If the stroke detection application 106 finds the facial drooping factor in the user 102 along with the speech slur factor in the voice of the user 102, and the numbness factor in hands of the user 102, then the stroke detection application 106 sends a notification to the emergency contact of the user 102. Otherwise, the stroke detection application 106 informs the user 102 that the symptoms of a stroke are not detected.

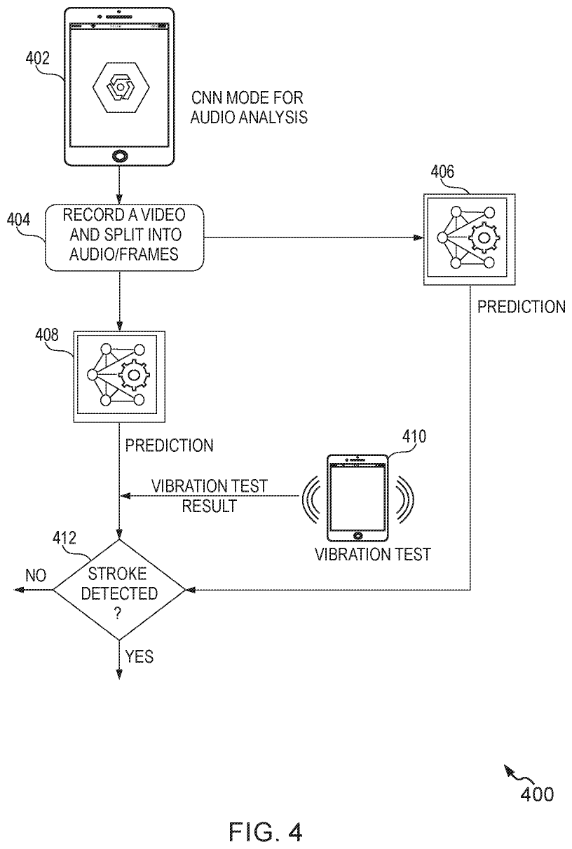

FIG. 4 is a simplified data flow diagram representation 400 for performing stroke detection using the first technique of the one or more techniques, in accordance with an embodiment of the present disclosure. It should be appreciated that each operation explained in the representation 400 is performed by the stroke detection application 106. The sequence of operations of the representation 400 may not be necessarily executed in the same order as they are presented. Further, one or more operations may be grouped and performed in form of a single step, or one operation may have several sub-steps that may be performed in parallel or a sequential manner. It is to be noted that to explain the process steps of FIG. 4, references may be made to system elements of FIG. 1 and FIG. 2.

At 402, the stroke detection application 106 utilizes a convolutional neural network model for performing audio analysis and face analysis of the user 102 for performing the first test using the first technique of the one or more techniques. In one embodiment, the stroke detection application 106 uses transfer learning for creating the convolutional neural network model.

At 404, the stroke detection application 106 displays instructions to the user 102 to record a video in the user device 104. In addition, the stroke detection application 106 splits the video into audio samples and images of the face of the user 102 in real-time.

At 406, the stroke detection application 106 utilizes the convolutional neural network to detect the speech slur factor in the voice of the user 102 recorded in the video in real-time. The stroke detection application 106 detects the speech slur factor as part of the first test being performed on the real-time video of the user 102 received through the user device 104.

At 408, the stroke detection application 106 utilizes the convolutional neural network to detect the facial drooping factor in face of the user 102 recorded in the video in real-time. The stroke detection application 106 detects the facial drooping factor as part of the first test being performed on the real-time video of the user 102 received through the user device 104.

At 410, the stroke detection application 106 performs the second test. The second test is the vibration test that is performed on the user device 104 to detect the numbness factor in the hands of the user 102.

At 412, the stroke detection application 106 processes the facial drooping factor, the speech slur factor, and the numbness factor to detect symptoms of the stroke present in the user 102. If the stroke detection application 106 finds the facial drooping factor in the user 102 along with the speech slur factor in the voice of the user 102, and the numbness in hands of the user 102, the stroke detection application 106 sends a notification to the emergency contact of the user 102. Otherwise, the stroke detection application 106 informs the user 102 that the symptoms of a stroke are not detected.

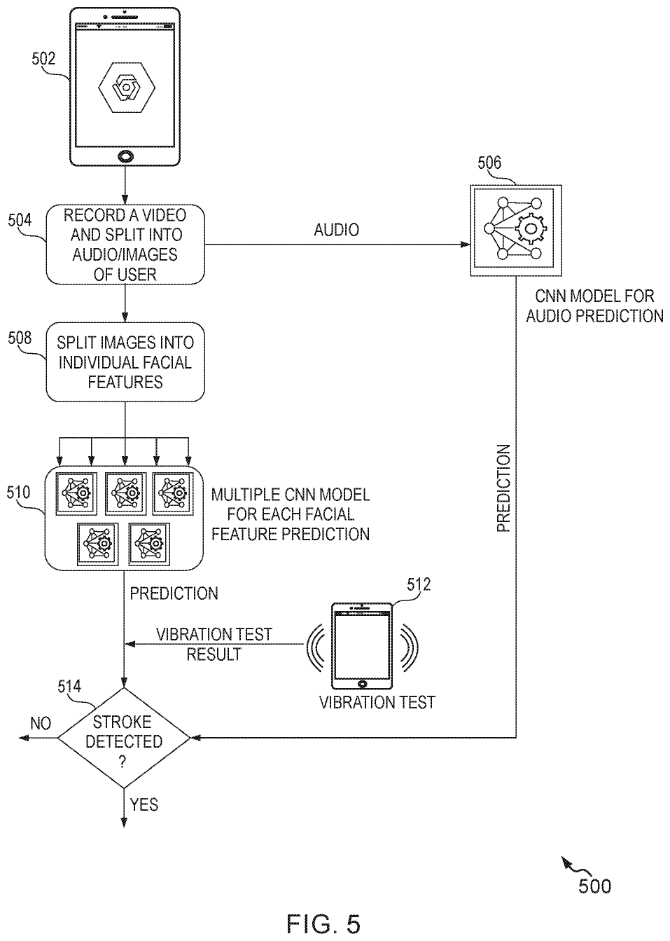

FIG. 5 is a simplified data flow diagram representation 500 for performing stroke detection using the second technique of the one or more techniques, in accordance with an embodiment of the present disclosure. It should be appreciated that each operation explained in the representation 500 is performed by the stroke detection application 106. The sequence of operations of the representation 500 may not be necessarily executed in the same order as they are presented. Further, one or more operations may be grouped and performed in form of a single step, or one operation may have several sub-steps that may be performed in parallel or in a sequential manner. It is to be noted that to explain the process steps of FIG. 5, references may be made to system elements of FIG. 1 and FIG. 2.

At 502, the stroke detection application 106 utilizes a convolutional neural network model for performing audio analysis and face analysis for performing the first test using the second technique of the one or more techniques. In one embodiment, the stroke detection application 106 uses transfer learning for creating the convolutional neural network model.

At 504, the stroke detection application 106 displays instructions to the user 102 to record a video in the user device 104. In addition, the stroke detection application 106 splits the video into audio samples and images of the face of the user 102 in real-time.

At 506, the stroke detection application 106 utilizes the convolutional neural network to detect the speech slur factor in the voice of the user 102 recorded in the video in real-time. The stroke detection application 106 detects the speech slur factor as part of the first test being performed on the real-time video of the user 102 received through the user device 104.

At 508, the stroke detection application 106 segments the face of the user 102 recorded in the accessed video into the plurality of facial segments in real-time. Each of the plurality of facial segments represents an individual face feature of the face of the user 102. In an example, the plurality of facial segments includes, but may not be limited to, right-left eyes, right-left eyebrows, lips, and jawline.

At 510, the stroke detection application 106 utilizes a plurality of convolutional neural networks to detect the facial drooping factor in face of the user 102 in real-time. Each of the plurality of convolutional networks is utilized for detection of the facial drooping factor in a particular facial segment of the plurality of facial segments. In one embodiment, the stroke detection application 106 utilizes the deep learning model to perform prediction of the facial drooping of the user 102 using the video received from the user device 104. The stroke detection application 106 detects the facial drooping factor as part of the first test being performed on the real-time video of the user 102 received through the user device 104.

At 512, the stroke detection application 106 performs the second test. The second test is the vibration test that is performed on the user device 104 to detect the numbness factor in hands of the user 102.

At 514, the stroke detection application 106 processes the facial drooping factor, the speech slur factor, and the numbness factor to detect symptoms of stroke in the user 102. If the stroke detection application 106 detects the facial drooping factor in the user 102 along with the speech slur factor in the voice of the user 102, and the numbness factor in hands of the user 102, the stroke detection application 106 sends a notification to the emergency contact of the user 102. Otherwise, the stroke detection application 106 informs the user 102 that the symptoms of stroke are not detected.

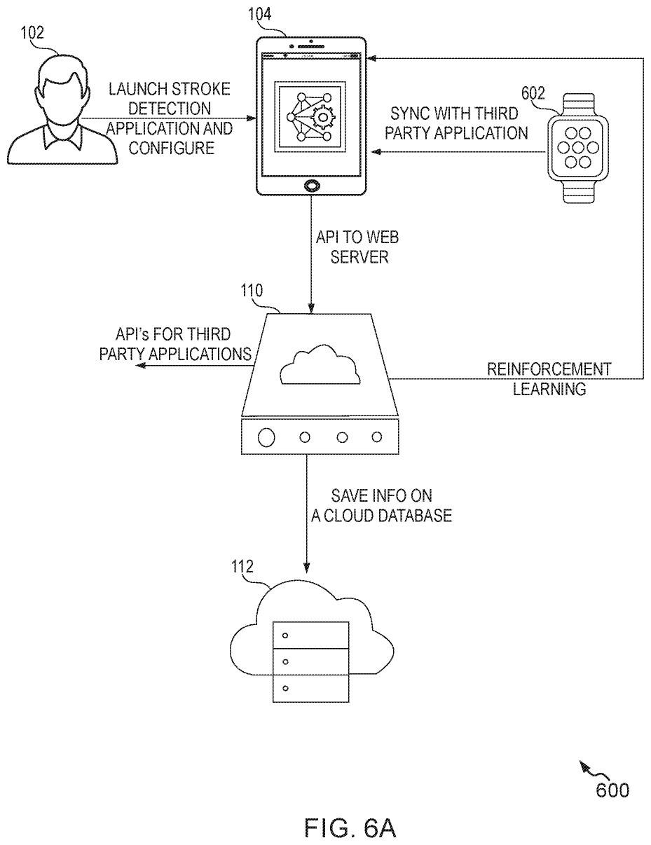

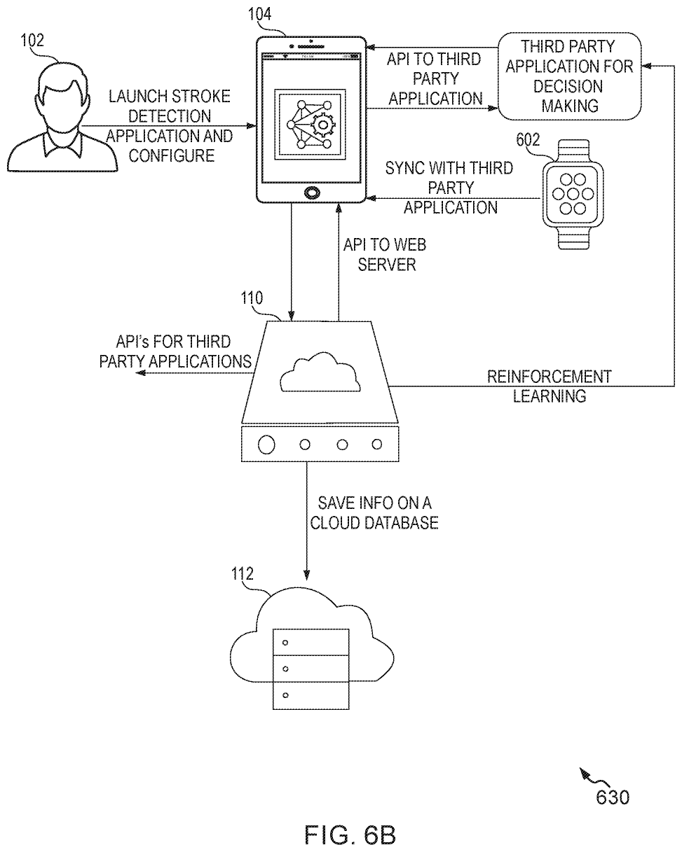

FIG. 6A is a high-level data flow diagram representation 600 for performing stroke detection using the first technique and the second technique of the one or more techniques, in accordance with an example embodiment of the present disclosure. FIG. 6B is a high-level data flow diagram representation 630 for performing stroke detection using the third technique of the one or more techniques, in accordance with an example embodiment of the present disclosure It is to be noted that to explain the process steps of FIG. 6A and FIG. 6B, references will be made to the system elements of FIG. 1 and FIG. 2.

In FIG. 6A and FIG. 6B, the user 102, the user device 104, a wearable device 602, the server system 110 and the database 112 are shown. The user 102 launches or configures the stroke detection application 106 in the user device 104. The user device 104 is associated with the user 102. In one embodiment, the user 102 is the owner of the user device 104.

The user 102 may download the stroke detection application 106 in the user device 104. The user 102 may use the network 108 such as internet, intranet, mobile data, wi-fi connection, 3G/4G/5G and the like to download the stroke detection application 106 in the user device 104. The user 102 operates the user device 104 to access the stroke detection application 106. In an example, the user device 104 includes, but may not be limited to, desktop, workstation, smart phone, tablet, laptop and personal digital assistant.

In an example, the user device 104 is an Android®-based smartphone. In another example, the user device 104 is an iOS-based iPhone. In yet another example, the user device 104 is a Windows®-based laptop. In yet another example, the user device 104 is a mac® OS-based MacBook. In yet another example, the user device 104 is a computer device running on any other operating system such as Linux®, Ubuntu®, Kali Linux®, and the like. In yet another example, the user device 104 is a mobile device running on any other operating system such as Windows, Symbian, Bada, and the like.

In one embodiment, the user 102 downloads the stroke detection application 106 on the user device 104. In another embodiment, the user 102 accesses the stroke detection application 106 on the user device 104 using a web browser installed on the user device 104. In an example, the web browser includes, but may not be limited to, Google Chrome®, Microsoft Edge®, Brave browser, Mozilla Firefox®, and Opera browser®.

The user device 104 connects with the wearable device 602 worn by the user 102. In general, wearable devices are smart electronic devices that are worn on or near body of the user 102 to track important biometric information related to the health or fitness of the user 102. In an example, the wearable device 602 includes, but may not be limited to, smart watch, fitness tracker, augmented reality-based headsets, and artificial intelligence-based hearing aids. In one embodiment, the third-party application is installed in the wearable device 602. The stroke detection application 106 synchronizes data with the third-party application installed inside the wearable device 602. In one embodiment, the wearable device 602 transmits additional health information of the user 102 to the user device 104 in real-time through the stroke detection application 106.

Referring now to FIG. 6A, the stroke detection application 106 utilizes the machine learning algorithms to perform the stroke detection in real-time. In one embodiment, the stroke detection application 106 utilizes the first technique of the one or more techniques to perform the first test. The stroke detection application 106 recognizes entire face of the user 102 to detect the facial drooping factor in real-time image of face of the user 102 using the machine learning algorithms (based on the convolutional neural network).

In another embodiment, the stroke detection application 106 utilizes the second technique of the one or more techniques to perform the first test. The stroke detection application 106 segments the entire face of the user 102 into the plurality of facial segments to improve the accuracy of detection of the facial drooping factor. Further, each of the plurality of facial segments is analyzed by the plurality of convolutional neural networks to detect the facial drooping factor in face of the user 102 in real-time using the deep learning algorithms (based on the plurality of convolutional neural networks).

In addition, the stroke detection application 106 utilizes the machine learning algorithms to detect the speech slur factor in the recorded audio of the user 102 extracted from the accessed video of the user 102 on the user device 104. Further, the stroke detection application 106 performs the second test (the vibration test) to detect the numbness factor in hands of the user 102 in real-time. Based on the processing of the facial drooping factor, the speech slur factor, and the numbness factor, the stroke detection application 106 detects whether the symptoms of a stroke are present in the user 102 or not.

Referring now to FIG. 6B, the stroke detection application 106 utilizes the third technique of the one or more techniques to perform stroke detection in real-time. In addition, the stroke detection application 106 records video (image samples) and audio (voice samples) of the user 102 at an initial stage as part of the calibration process when the user 102 launches the stroke detection application 106 for the first time in the user device 104. The recorded video and audio of the user 102 are stored in the database 112.

The user 102 launches the stroke detection application 106 if the user 102 feels symptoms of the stroke. In one example, symptoms of stroke include numbness, difficulty in balancing and walking, difficulty in breathing, trouble walking, vision problems, dizziness, and the like. The stroke detection application 106 displays instructions on a display of the user device 104 to notify the user 102 to record the video in camera of the user device 104. The stroke detection application 106 also displays instructions on the display of the user device 104 to notify the user 102 to speak a specific phrase in the video being recorded in the camera of the user device 104 in real-time. In an example, the specific phrase may be “The prospect of cutting back spending is an unpleasant one of any governor”. However, the specific phrase is not limited to above-mentioned phrase.

The stroke detection application 106 compares the face of the user 102 recorded in the video in the user device 104 in real-time with the face of the user 102 already stored in the database 112 in the initial step as part of the calibration process. The stroke detection application 106 performs the comparison to detect the facial drooping factor of the user 102 in real-time.

Similarly, the stroke detection application 106 compares the audio of the user 102 recorded in the video in the user device 104 in real-time with the audio (voice samples) of the user 102 already stored in the recorded video in the database 112 in the initial step as part of the calibration process. The stroke detection application 106 performs the comparison to detect the speech slur factor of the user 102 in real-time. Further, the stroke detection application 106 performs the second test (the vibration test) to detect the numbness factor in hands of the user 102 in real-time. Based on the processing of the facial drooping factor, the speech slur factor, and the numbness factor, the stroke detection application 106 detects whether symptoms of a stroke are present in the user 102 or not.

The stroke detection application 106 utilizes an API to connect to the server system 110. In one embodiment, the stroke detection application 106 is associated with the server system 110. In another embodiment, the stroke detection application 106 is installed at the server system 110. The server system 110 handles each operation and task performed by the stroke detection application 106. The server system 110 stores one or more instructions and one or more processes for performing various operations of the stroke detection application 106. In one embodiment, the server system 110 is a cloud server. In general, cloud server is built, hosted, and delivered through a cloud computing platform. In general, cloud computing is a process of using remote network servers that are hosted on the internet to store, manage, and process data. In one embodiment, the server system 110 includes APIs to connect with other third-party applications (as shown in FIG. 6A and FIG. 6B).

In one example, the other third-party applications include pharmacy applications. In another example, the other third-party applications include insurance applications. In yet another example, the other third-party applications include hospital applications connected with various hospitals, blood sugar applications, and the like.

The server system 110 includes the database 112. The database 112 is used for storage purposes. The database 112 is associated with the server system 110. In general, database is a collection of information that is organized so that it can be easily accessed, managed, and updated. In one embodiment, the database 112 provides storage location to all data and information required by the stroke detection application 106. In one embodiment, the database 112 is a cloud database. In another embodiment, the database 112 may be at least one of hierarchical database, network database, relational database, object-oriented database and the like. However, the database 112 is not limited to the above-mentioned databases.

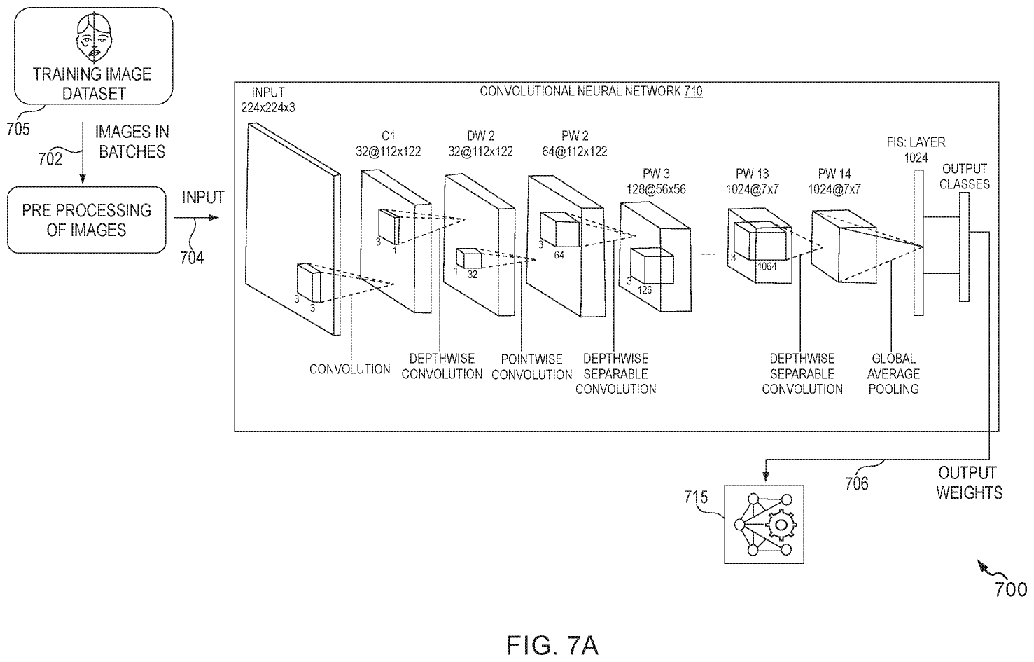

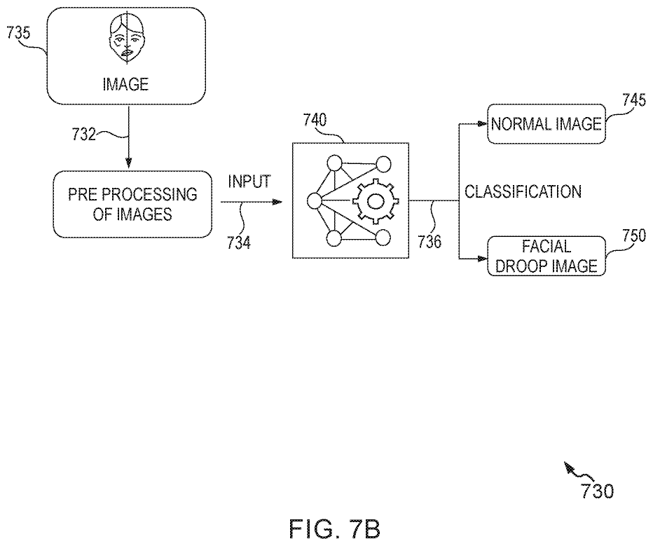

FIG. 7A is a schematic representation 700 of a process for training a deep learning model for detecting facial drooping factor, in accordance with an embodiment of the present disclosure. The schematic representation 700 is explained herewith including entities such as, a training image dataset 705, a convolutional neural network 710, and a deep learning model 715. The deep learning model 715 may include the plurality of machine learning models.

As mentioned previously, the stroke detection application 106 is trained to detect the facial drooping factor in face of the user 102. In other words, a deep learning model (e.g., the deep learning model 715) is trained to detect the facial drooping factor in face of the user 102. As shown in FIG. 7A, the training image dataset 705 includes various facial images of multiple users to train the deep learning model 715. In one embodiment, the training image dataset 705 includes the sample facial data sets of non-facial muscle drooped images (i.e., normal images) and facial muscle drooped images (i.e., disease state images) to train the deep learning model 715. The deep learning model 715 is trained with the training image dataset 705 to accurately differentiate between the normal face image of the user 102 and the facial droop image of the user 102.

Before training the deep learning model 715, images present in the training image dataset 705 undergoes data pre-processing operations in batches (see, 702). The data pre-processing operations may be performed to extract features from the various facial images of the multiple users. In one embodiment, the data pre-processing operations may include morphological transformations, de-noising, normalization, and the like.

Upon completion of the data pre-processing operations, the training image dataset 705 is fed as an input to the convolutional neural network 710 (see, 704). In general, convolutional neural network (CNN) is a type of artificial neural network usually applied for the analysis of visual data (e.g., images). More specifically, CNN is an algorithm that receives an image file as an input, assigns parameters (e.g., weights and biases) to various aspects in the image file, to be able to differentiate the image file from other images.

Based on the processing of the convolutional neural network 710, the deep learning model 715 is trained (see, 706). In one embodiment, the deep learning model 715 is trained based on output weights calculated by the convolutional neural network 710. In some embodiments, the deep learning model 715 is trained based on transfer learning. In general, transfer learning is a machine learning technique in which knowledge gained while solving one problem is stored and further applied to a different but related problem. In other words, a model developed for a task may be reused as a starting point for another model on a second task.

In general, transfer learning is a commonly used deep learning approach where pre-trained models are used as a starting point in computer vision and natural language processing (NLP) tasks, because of the vast compute and time resources required to develop such NN models and from the huge jumps in performance metrics that they provide on related problems. In some embodiments, transfer learning may be used to train a deep learning model (e.g., the deep learning model 715).

For example, to train any deep learning model with transfer learning, a related predictive modeling problem must be selected with scalable data showing at least some relationship in input data, output data, and/or concepts learned during mapping from the input data to output data. Thereafter, a source model must be developed for performing a first task. Generally, this source model must be better than a naive model to ensure that feature learning has been performed. Further, fit of the source model on source task may be used as a starting point for a second model on second task of interest. This may include using all or parts of the source model based, at least in part, on the modeling technique used. Alternatively, the second model may need to be adapted or refined based on the input-output pair data available for the task of interest.

FIG. 7B is a schematic representation 730 of a process for implementation of the deep learning model for detecting the facial drooping factor in real-time, in accordance with an embodiment of the present disclosure. The schematic representation 730 is explained herewith including entities such as, an image 735, a deep learning model 740, a normal image 745, and facial droop image 750.

As explained above, the stroke detection application 106 is configured to execute the deep learning model (e.g., the deep learning model 740) to detect the facial drooping factor in face of the user 102 in real-time. For detecting the facial drooping factor, real-time video or image (i.e., the image 735) of the user 102 is captured through the camera (i.e., either front-facing camera or back camera) of the user device 104 of the user 102. The image 735 further undergoes pre-processing operations such as morphological transformations, de-noising, normalization, and the like (see, 732).

Once the pre-processing operations on the image 735 are complete, the image 735 is fed as an input to the deep learning model 740 (see, 734). In one embodiment, the deep learning model 740 is trained version of the deep learning model 715. The pre-trained deep learning model (i.e., the deep learning model 740) is used to perform image classification in real-time to classify the image 735 as either the normal image 745 or the facial droop image 750 (see, 736). In one embodiment, the deep learning model 740 is integrated with the stroke detection application 106 to detect the facial drooping in face of the user 102 in real-time.

In some embodiments, transfer learning may be used on a pre-trained deep learning (DL) model. For example, a pre-trained DL model is selected or chosen from various available DL models. In one example, DL models may be timely released by facilities (e.g., companies, organizations, research institutions, etc.) based on large and challenging datasets. The pre-trained DL model may be used as a starting point for a second model on the second task of interest. This may include using all or parts of the pre-trained DL model based, at least in part, on the modeling technique used. Alternatively, the second model may need to be adapted or refined based on the input-output pair data available for the task of interest.

In one example, the deep learning model 740 is created based on MobileNet architecture. In general, MobileNet is a mobile computer vision model designed to be used in mobile applications. In addition, MobileNet architecture uses depth-wise separable convolutions that significantly reduce the number of parameters when compared to a network with regular convolutions with the same depth in the nets. This further results in lightweight DNNs. Generally, a depth-wise separable convolution may be created from two operations, namely depth-wise convolution and pointwise convolution. Further, the architecture of MobileNet model is illustrated below in Table 1:

TABLE 1
Architecture of MobileNet model

| Type/Stride | Filter Shape | Input Size |
|---|---|---|
| Conv/s2 | 3 × 3 × 3 × 32 | 224 × 224 × 3 |
| Conv dw/s1 | 3 × 3 × 32 dw | 112 × 112 × 32 |
| Conv/s1 | 1 × 1 × 32 × 64 | 112 × 112 × 32 |
| Conv dw/s2 | 3 × 3 × 64 dw | 112 × 112 × 64 |
| Conv/s1 | 1 × 1 × 64 × 128 | 56 × 56 × 64 |
| Conv dw/s1 | 3 × 3 × 128 dw | 56 × 56 × 128 |
| Conv/s1 | 1 × 1 × 128 × 128 | 56 × 56 × 128 |
| Conv dw/s2 | 3 × 3 × 128 dw | 56 × 56 × 128 |
| Conv/s1 | 1 × 1 × 128 × 256 | 28 × 28 × 128 |
| Conv dw/s1 | 3 × 3 × 256 dw | 28 × 28 × 256 |
| Conv/s1 | 1 × 1 × 256 × 256 | 28 × 28 × 256 |
| Conv dw/s2 | 3 × 3 × 256 dw | 28 × 28 × 256 |
| Conv/s1 | 1 × 1 × 256 × 512 | 14 × 14 × 256 |
| 5×_{Conv/s1}^{Conv dw/s1} | 3 × 3 × 512 dw | 14 × 14 × 512 |
| | 1 × 1 × 512 × 512 | 14 × 14 × 512 |
| Conv dw/s2 | 3 × 3 × 512 dw | 14 × 14 × 512 |
| Conv/s1 | 1 × 1 × 512 × 1024 | 7 × 7 × 512 |
| Conv dw/s2 | 3 × 3 × 1024 dw | 7 × 7 × 1024 |
| Conv/s1 | 1 × 1 × 1024 × 1024 | 7 × 7 × 1024 |
| Avg Pool/s1 | Pool 7 × 7 | 7 × 7 × 1024 |
| FC/s1 | 1024 × 1000 | 1 × 1 × 1024 |
| Softmax/s1 | Classifier | 1 × 1 × 1000 |

In some embodiment, the deep learning model 740 is converted into TensorFlow Lite (TFLite) format for successful integration with the stroke detection application 106. In addition, the TFLite format of the deep learning model 740 may be integrated with the stroke detection application 106 installed in the user device 104 running on any operating system (e.g., iOS, Android, Windows, Bada, Symbian, Blackberry, etc.). In some embodiments, the deep learning model 740 is trained with the facilitation of transfer learning and MobileNet architecture. The deep learning model 740 further achieved an accuracy of 86% on the training data set and an accuracy of 96.67% on the validation data set. In one embodiment, weights of the deep learning model 740 with the aforementioned accuracy are stored and converted into TFLite format for integration with the stroke detection application 106.

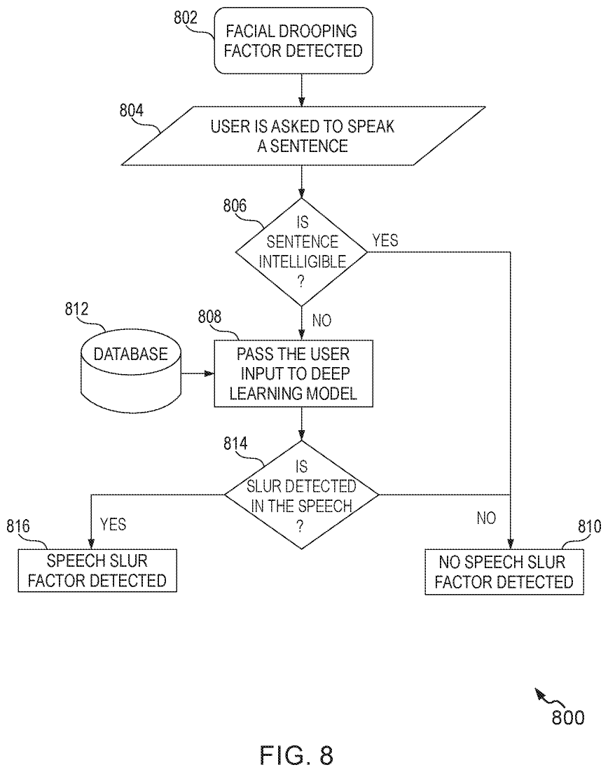

FIG. 8 is a simplified data flow diagram representation for detecting speech slur factor in the voice of the user 102 in real-time, in accordance with an embodiment of the present disclosure. It should be appreciated that each operation explained in the representation 800 is performed by the stroke detection application 106. The sequence of operations of the representation 800 may not be necessarily executed in the same order as they are presented. Further, one or more operations may be grouped together and performed in form of a single step, or one operation may have several sub-steps that may be performed in parallel or a sequential manner. It is to be noted that to explain the process steps of FIG. 8, references may be made to system elements of FIG. 1 and FIG. 2.

At 802, the stroke detection application 106 detects the facial drooping factor in face of the user 102. Upon detection of the facial drooping factor, the stroke detection application 106 detects the speech slur factor in the voice of the user 102.

At 804, the stroke detection application 106 asks the user to record audio or voice through the microphone of the user device 104. In some embodiments, the stroke detection application 106 may display a command in user interface (UI) of the stroke detection application 106 requesting the user 102 to speak a specific sentence or paragraph. In one embodiment, the stroke detection application 106 may ask the user 102 to speak the specific sentence or paragraph (as displayed on the screen of the user device 104) loud and clear. In one embodiment, the stroke detection application 106 may record the audio of the user 102 while capturing the face of the user 102 during video recording performed for detecting the facial drooping factor.

At 806, the stroke detection application 106 checks whether the recorded voice or audio of the user 102 is intelligible (i.e., easily understandable, or interpretable) or not. If the recorded audio of the user 102 is intelligible, at 810, the stroke detection application detects no speech slur factor in the voice of the user 102.

If the recorded audio of the user 102 is not intelligible, at 808, the stroke detection application 106 passes the recorded audio of the user 102 to the deep learning model 740 (e.g., pre-trained deep learning model) to detect the speech slur factor in voice of the user 102. At 812, the stroke detection application 106 may query the database 112 to access the already recorded and stored voice of the user 102 in the database 112.

At 814, the stroke detection application 106 compares the audio of the user 102 captured in real-time with the audio of the user 102 already stored in the database 112. In one embodiment, the stroke detection application 106 may perform the comparison with the execution of the machine learning model or the deep learning model. The stroke detection application 106 performs the comparison to detect whether the speech slur factor is present in voice of the user 102 or not. In one embodiment, the comparison may be performed to detect whether any anomalies are present in voice of the user 102. Based on the comparison, the stroke detection application 106 may classify voice of the user as either normal voice (i.e., non-audio slur) or speech slur voice (i.e., disease state). If anomalies are not present in voice of the user 102, at 810, the stroke detection application 106 detects no speech slur factor in voice of the user 102 and classifies the voice as normal voice. Otherwise, at 816, the stroke detection application 106 detects the speech slur factor in voice of the user 102 in real-time and classifies the voice as speech slur voice.

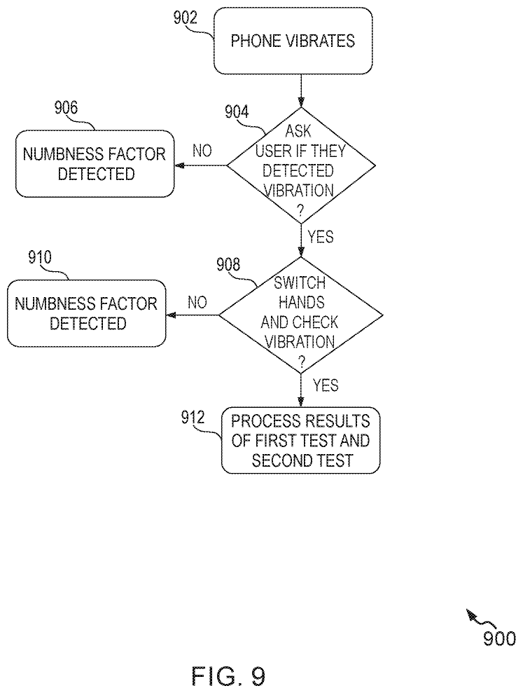

FIG. 9 is a simplified data flow diagram representation for detecting numbness factor in hands of the user in real-time, in accordance with an embodiment of the present disclosure. It should be appreciated that each operation explained in the representation 900 is performed by the stroke detection application 106. The sequence of operations of the representation 900 may not be necessarily executed in the same order as they are presented. Further, one or more operations may be grouped together and performed in form of a single step, or one operation may have several sub-steps that may be performed in parallel or in a sequential manner. It is to be noted that to explain the process steps of FIG. 9, references may be made to system elements of FIG. 1 and FIG. 2.

At 902, the stroke detection application 106 may interact with vibration hardware of the user device 104 to vibrate the user device 104. In some embodiments, the stroke detection application 106 may vibrate the user device 104 in some patterns along with pauses in between. In one embodiment, the stroke detection application 106 may provide UI on the user device 104 to allow the user 102 to adjust level of vibration.

At 904, the stroke detection application 106 asks the user 102 whether any vibration is detected by the user 102 or not. In one embodiment, UI of the stroke detection application 106 may display instructions to the user 102 asking whether the user 102 felt vibration in the user device 104 or not. The user 102 may further tap/click/press on a yes button if the user 102 felt the vibration or the user 102 may tap on the no button if vibration is not felt by the user 102.

At 906, the stroke detection application 106 may detect the numbness factor in hands of the user 102 if the user 102 accepts that the vibration of the user device 104 has not been felt by the user 102. In one example, the user 102 may click/press/tap on the no button to accept that vibration in the user device 104 has not been felt by the user 102.

If the user 102 feels vibration in the user device 104, at 908, the stroke detection application 106 may ask the user 102 to switch hands and then again perform the second test (i.e., vibration test for numbness factor detection) for confirmation. In an example, if the user 102 is holding the user device 104 in right hand, the stroke detection application 106 may display instructions to the user 102 to hold the user device 104 in left hand and perform the second test again. In another example, if the user 102 is holding the user device 104 in left hand, the stroke detection application 106 may display instructions to the user 102 to hold the user device 104 in right hand and perform the second test again. The user 102 may further tap/click/press on a yes button if the user 102 felt the vibration or the user 102 may tap on the no button if the vibration is not felt by the user 102.

If the user 102 accepts that the vibration of the user device 104 has not been felt by the user 102, at 910, the stroke detection application 106 may detect the numbness factor in hands of the user 102. In one example, the user 102 may click/press/tap on the no button to accept that vibration in the user device 104 has not been felt by the user 102. In such scenario, the stroke detection application 106 may send a notification to at least one emergency contact of the user 102 for providing medical assistance to the user 102. Otherwise, at 912, the stroke detection application 106 may process the results of the first test and further based on processing of results of the first test and the second test, the stroke detection application 106 may detect whether the symptoms of stroke are detected in the user 102 or not.

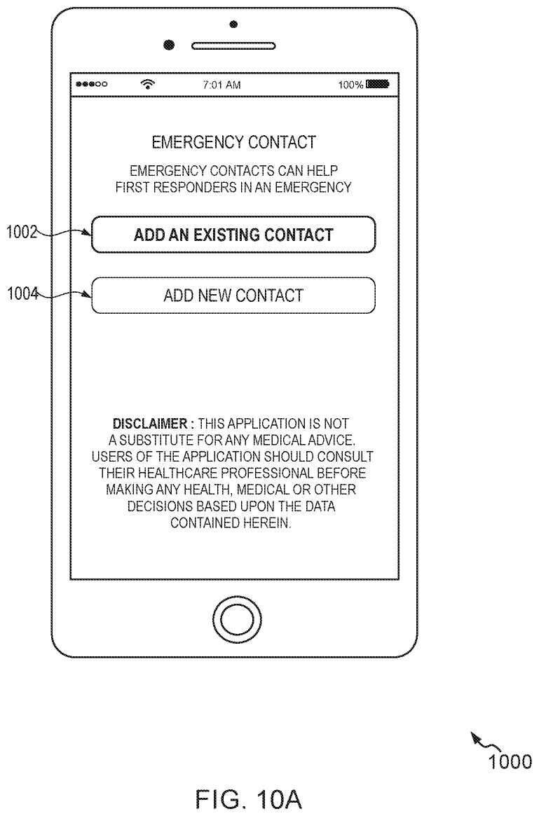

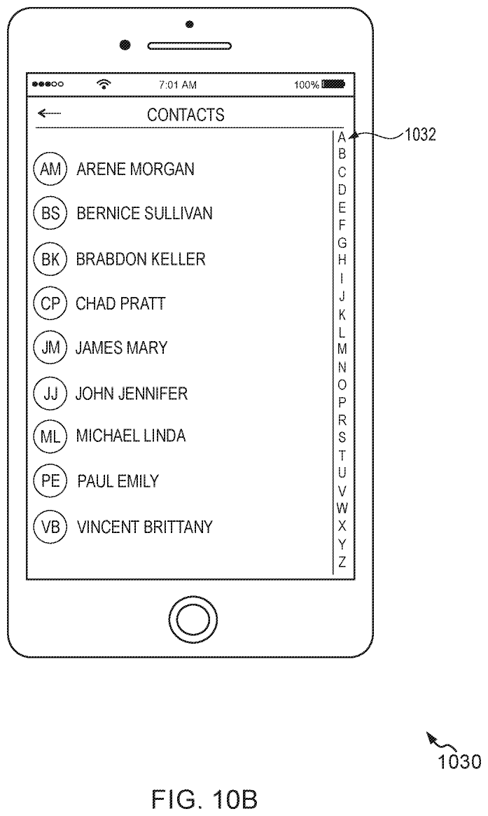

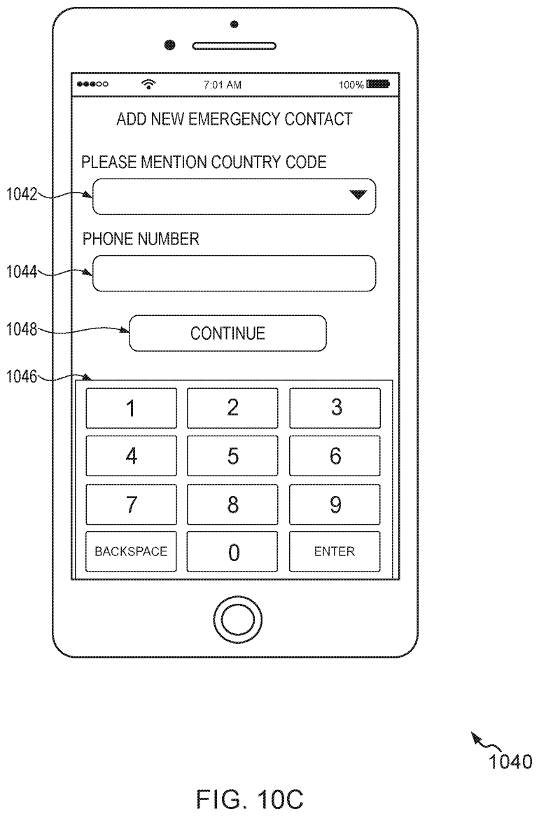

FIGS. 10A-10C, collectively, represent user interfaces (UIs) of application for setting up an emergency contact to notify in case symptoms of stroke are detected in the user 102, in accordance with an embodiment of the present disclosure. As mentioned earlier, the stroke detection application 106 sends a notification in real-time to the emergency contact of the user 102. The various UIs shown in the FIGS. 10A-10C depict process steps performed by the stroke detection application 106 to allow the user 102 to set the emergency contact of the user 102 through the stroke detection application 106. In one embodiment, the stroke detection application 106 stores information of the emergency contact in the database 112. In another embodiment, the stroke detection application 106 stores information of the emergency contact in the stroke detection application 106.

In the FIG. 10A, UI 1000 of a screen to add the emergency contact information is shown. The UI 1000 displays two buttons to add the emergency contact information. The two buttons include “Add an existing contact” button (see, 1002) and “Add new contact” button (see, 1004). The user 102 may click/tap/press on the “Add an existing contact” button to add a contact stored in the contact list of the user device 104 to the emergency contact list. Otherwise, the user 102 may click/tap/press on the “Add new contact” button to add a new contact that is not already stored in the contact list of the user device 104 to the emergency contact list.

In the FIG. 10B, UI 1030 of “Add an existing contact” page is shown. The UI 1030 is shown after the user 102 taps/clicks/presses the “Add an existing contact” button. The UI 1030 displays list of contacts that are already stored in the contact list of the user device 104. The user 102 may tap/click/press on any name in the contact list to set the contact as emergency contact of the user 102. The emergency contact of the user 102 is that contact whom the user 102 wishes to inform in case of medical emergency such as the stroke. In one embodiment, the user 102 may select any number of contacts as emergency contacts to be called or messaged in case the user 102 is detected with symptoms of stroke. The UI 1030 displays a slider 1032 on left side of screen of the user device 104 to easily scroll through the contact list alphabetically.

In the FIG. 10C, UI 1040 of “Add new contact” page is shown. The UI 1040 is shown after the user 102 taps/clicks/presses the “Add new contact” button. The “Add new contact” page displays a drop-down list (see, 1042) to select country code of the emergency contact of the user 102. The user 102 may tap/click/press the drop-down list to view a list of all the available country codes. In an example, the user 102 selects “United States (+1)” if the emergency contact of the user 102 belongs to United States of America. In another example, the user 102 selects “India (+91)” if the emergency contact of the user 102 belongs to India.

Further, the UI 1040 displays a text box (see, 1044) to allow the user 102 to enter phone number of the emergency contact in the text box. When the user 102 press/taps/clicks on the text box, a dialer 1046 pops up on the screen of the user device 104 that allows the user 102 to type the phone number of the emergency contact. Furthermore, the user 102 may tap/click/press on “Continue” button (see, 1048) to save the phone number as an emergency contact of the user 102. In one embodiment, the user 102 may add any number of contacts as emergency contacts to be contacted in case the user 102 is detected with symptoms of stroke.

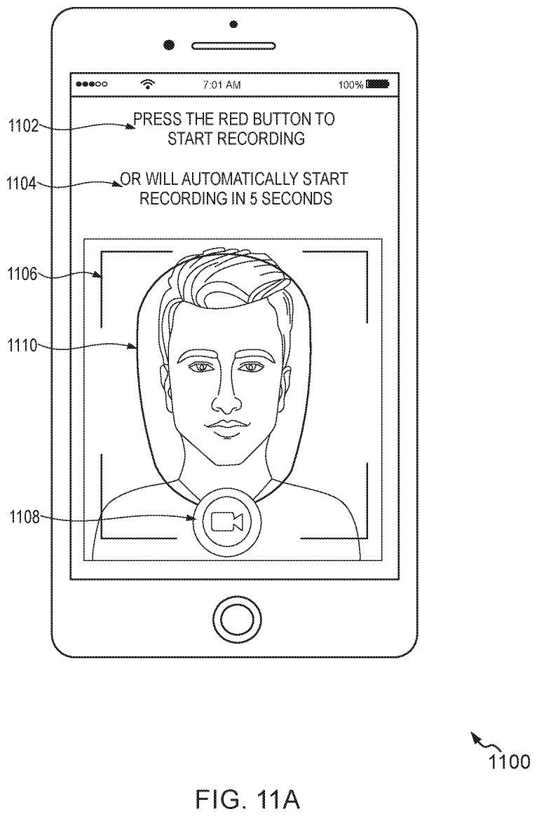

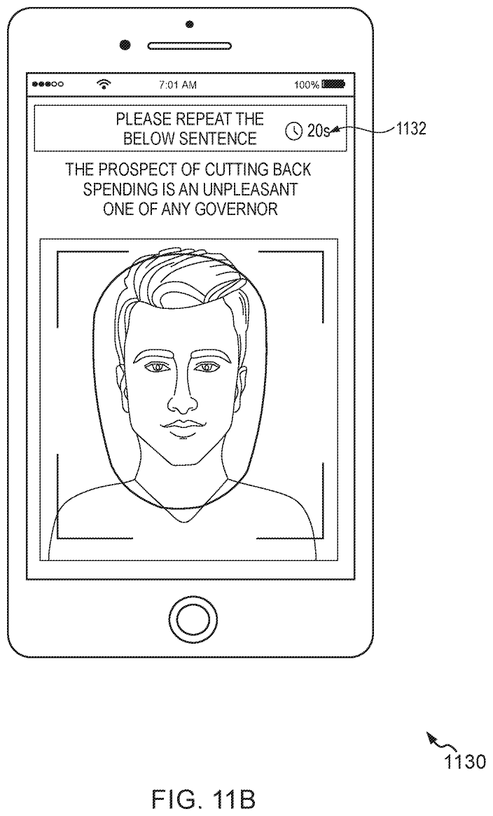

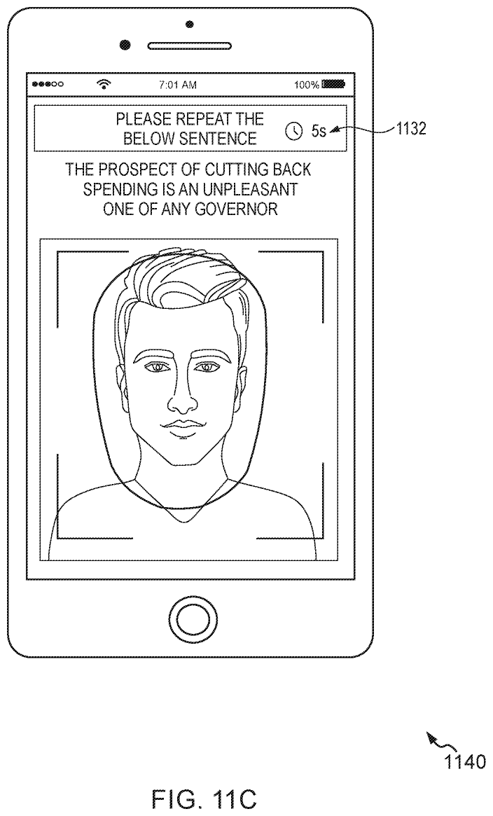

FIGS. 11A-11C, collectively, represent user interfaces (UIs) of the application for performing the first test for performing stroke detection, in accordance with an embodiment of the present disclosure. As mentioned earlier, the stroke detection application 106 performs the first test in real-time to perform stroke detection. The first test includes detecting the facial drooping factor and the speech slur factor in real-time. The various UIs shown in the FIGS. 11A-11C depict process steps performed by the stroke detection application 106 to perform the first test in real-time.

In the FIG. 11A, UI 1100 of the screen to display instructions to the user 102 to record the video of the user 102 in real-time to perform the first test is shown. The UI 1100 displays text “Press the red button to start recording” (see, 1102) to provide instructions to the user 102 to press/click/tap on the red button displayed on the bottom of the screen of the user device 104 to initialize the first test. Below the text “Press the red button to start recording”, the UI 1100 displays another text “or will automatically start recording in 5 seconds . . . ” (see, 1104) to inform the user 102 that otherwise the recording will start automatically in 5 seconds. 5 seconds depict a timer of 5 seconds after which the stroke detection application 106 starts recording video of the user 102.

Below the text “or will automatically start recording in 5 seconds . . . ”, the stroke detection application 106 displays a camera viewfinder (see, 1106) depicting the real-time video being captured through the user device 104. In one embodiment, the stroke detection application 106 opens front-facing camera of the user device 104 to record the video of the user 102 in real-time. In another embodiment, the stroke detection application 106 opens the back camera of the user device 104 to record the video of the user 102 in real-time.

Further, the circular red button with video symbol (see, 1108) overlaps the camera viewfinder as shown in the UI 1100. The user 102 may click/press/tap on the red button to start the video recording real-time video of the user 102. Otherwise, the video recording may start after the timer of 5 seconds is complete. The real-time video of the user 102 is recorded to detect the facial drooping factor of the user 102. The facial drooping factor of the user 102 is detected using the one or more techniques discussed above. In addition, a white boundary (see, 1110) appears in the video recording that detects the face of the user 102 in the entire video being recorded through the camera of the user device 104.

In the FIG. 11B, UI 1130 of the screen to provide instructions to the user 102 to speak the specific phrase in the video of the user 102 being recorded in real-time to perform the first test is shown. After the user 102 clicks/presses/taps the circular red button or the stroke detection application 106 automatically starts the video recording, the UI 1130 displays the instructions “Please repeat the below sentence” to instruct the user 102 to speak the displayed specific phrase to detect the speech slur factor in the voice of the user 102.

On the right-hand side of the instructions, a timer (see, 1132) is shown. In one example, the timer is of 20 seconds. In another example, the timer is of 40 seconds. In yet another example, the timer is of 1 minute. However, the timer is not limited to above mentioned time. The user 102 has to speak the specific phrase in the video being recorded within the time interval of the timer.

Further, the UI 1130 displays the specific phrase (text) “The prospect of cutting back spending is an unpleasant one of any governor” for the user 102 to speak in the video being recorded in the camera of the user device 104. The user 102 speaks this specific phrase in the video being recorded in the user device 104. Furthermore, the stroke detection application 106 displays a camera viewfinder (see, 1106) depicting the real-time video being recorded through the user device 104. Moreover, a white boundary (see, 1110) appears in the video recording that detects face of the user 102 in the entire video being recorded through the camera of the user device 104.

In the FIG. 11C, UI 1140 of screen to provide instructions to the user 102 to speak the specific phrase in the video of the user 102 being recorded in real-time to perform the first test is shown. The UI 1140 displays the instructions “Please repeat the below sentence” to instruct the user 102 to speak the below displayed specific phrase to detect the speech slur factor in voice of the user 102.

On right-hand side of the instructions, the timer (see, 1132) is shown. In the FIG. 11C, the timer has changed to 5 seconds from prior timer of 20 seconds. In addition, color of the timer is changed to red color from black color to indicate that only 5 seconds are left for the user 102 to speak the specific phrase.

Further, the UI 1140 displays the specific phrase (text) “The prospect of cutting back spending is an unpleasant one of any governor” for the user 102 to speak in the video being recorded in the camera of the user device 104. The user 102 speaks this specific phrase in the video being recorded in the user device 104. Furthermore, the stroke detection application 106 displays a camera viewfinder (see, 1106) depicting the real-time video being recorded through the user device 104. Moreover, a white boundary (see, 1110) appears in the video recording that detects face of the user 102 in the entire video being recorded through the camera of the user device 104.

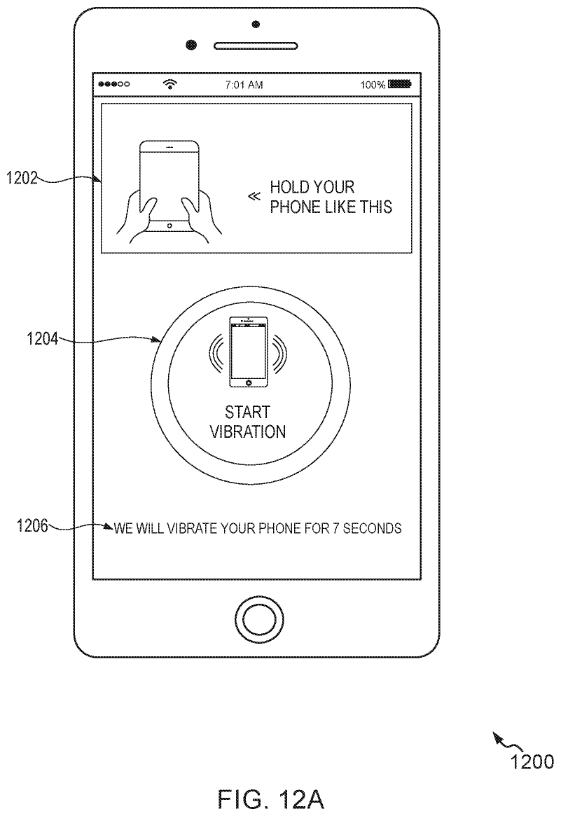

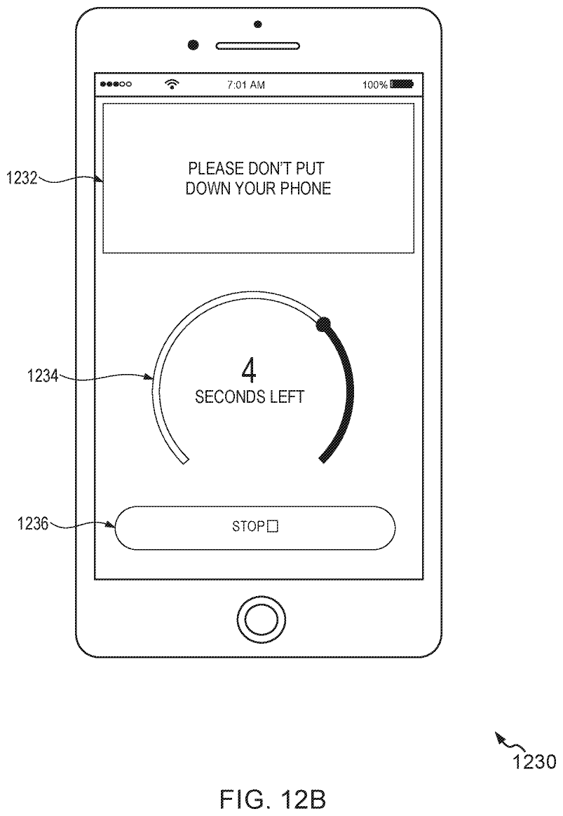

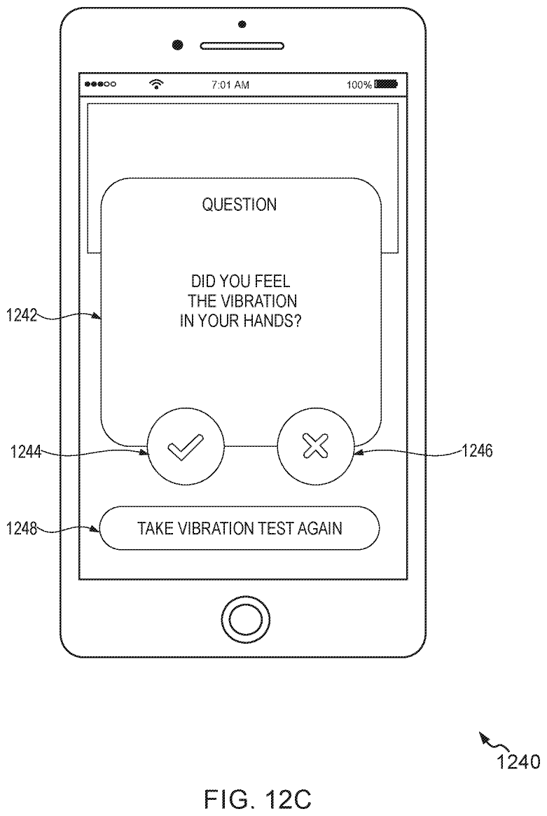

FIGS. 12A-12C, collectively, represent user interfaces (UIs) of application for performing the second test for stroke detection, in accordance with an embodiment of the present disclosure. As mentioned earlier, the stroke detection application 106 performs the second test in real-time to perform stroke detection. The second test is the vibration test performed to detect the numbness factor in hands of the user 102 in real-time. The various UIs shown in the FIGS. 12A-12C depict process steps performed by the stroke detection application 106 to perform the second test in real-time.

In the FIG. 12A, a UI 1200 to initialize the second test in the user device 104 is shown. The UI 1200 displays an icon (see, 1202) supporting the instructions (text) “Hold your phone like this” to instruct the user 102 to hold the user device 104 in a specific position as shown in the icon (see, 1202). In addition, the UI 1200 displays a circular button (see, 1204) with text “Start vibration”. Once the user 102 clicks/presses/taps on the circular button, the user device 104 starts vibrating for the second interval of time in real-time. In one example, the second interval of time is 7 seconds. In another example, the second interval of time is 15 seconds. However, the second interval of time is not limited to the above-mentioned time.

Further, the UI 1200 displays a text “We will vibrate your phone for 7 seconds” (see, 1206) to inform the user 102 that the user device 104 will be vibrated for 7 seconds after the user 102 clicks/presses/taps on the “Start vibration” button. The second interval of time for which the user device 104 vibrates may vary.

In the FIG. 12B, UI 1230 of the stroke detection application 106 in the middle of the second test is shown. After the user 102 clicks/presses/taps on the “Start vibration” button, the UI 1230 displays a warning “Please don't put down your phone” (see, 1232) to the user 102 to warn the user 102 not to put down the user device 104 as the second test is being performed by the stroke detection application 106 in real-time.

In addition, the UI 1230 displays the timer (see, 1234) being run in real-time in the stroke detection application 106. By default, the timer is of 7 seconds. Below the timer, the UI 1230 displays “Stop” button (see, 1236) to stop the timer in between. The user 102 may tap/click/press the “Stop” button if the user 102 wants to cancel or terminate the second test in between.

In the FIG. 12C, UI 1240 of a question screen that is displayed to the user 102 after completion of the second test is shown. The UI 1240 displays a question “Did you feel the vibration in your hands?” (see, 1242) asked from the user 102. The question is asked from the user 102 to detect the numbness factor in the hands of the user 102. If the user 102 felt the vibration through the user device 104, the user 102 may click/press/tap on the “Yes” button (see, 1244). Otherwise, the user 102 may click/press/tap on the “No” button (see, 1246) to inform the stroke detection application 106 that the user 102 did not feel any vibration. Based on the response received from the user 102, the stroke detection application 106 detects the numbness factor in the hands of the user 102.

The UI 1240 also displays a button with text “Take vibration test again” (see, 1248). The user 102 may click/press/tap this button to take up the second test again in real-time.

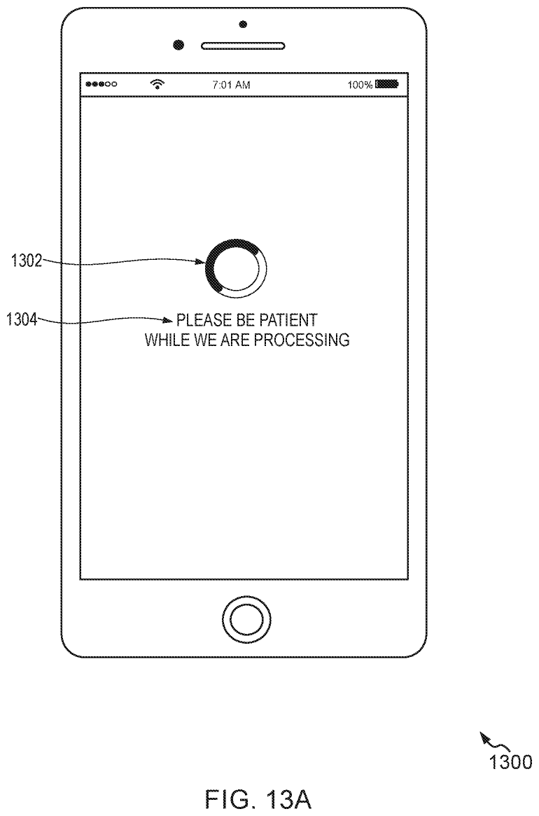

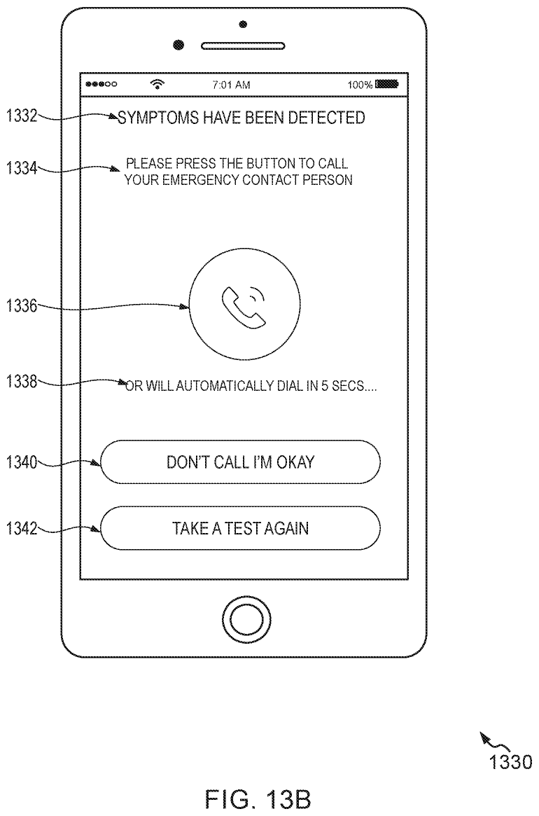

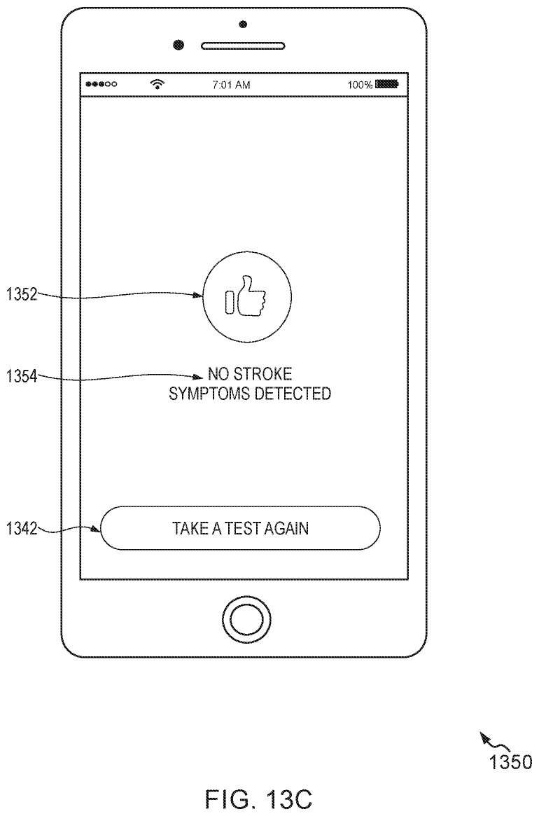

FIGS. 13A-13C, collectively, represent user interfaces (UIs) of application for processing results of the first test and the second test for performing stroke detection, in accordance with an embodiment of the present disclosure. As mentioned earlier, the stroke detection application 106 processes the facial drooping factor, the speech slur factor, and the numbness factor in real-time to detect symptoms of stroke in the user 102 in real-time. The various UIs shown in the FIGS. 13A-13C depict process steps performed by the stroke detection application 106 to process results of the first test and the second test in real-time.

In the FIG. 13A, UI 1300 depicting processing screen after performing the first test and the second test is shown. The UI 1300 displays circular processing icon (see, 1302) to show that the stroke detection application 106 is processing the results of the first test (the facial drooping factor and the speech slur factor) and the second test (the numbness factor). The UI 1300 also displays the text “Please be patient while we are processing . . . ” (see, 1304) to inform the user 102 to wait for the stroke detection application 106 to complete the processing and inform the user 102 whether the symptoms of stroke are detected by the stroke detection application 106 or not.

In the FIG. 13B, UI 1330 of a screen that appears if symptoms of stroke are detected in the user 102 is shown. The UI 1330 informs the user 102 with text “Symptoms has been detected” (see, 1332). In addition, the UI 1330 displays the text “Please press the button to call your emergency contact person” (see, 1334) to inform the user 102 to press the button to immediately call the emergency contact person.

Further, the UI 1330 displays a red button (see, 1336). The user 102 may press/click/tap on the red button to send notifications (for instance, call or message) to the emergency contact stored by the user 102 in the stroke detection application 106. Furthermore, the UI 1330 displays text “or will automatically dial in 5 secs . . . ” (see, 1338) to inform the user 102 that the stroke detection application 106 may automatically call the emergency contact in 5 seconds if the user 102 does not press/click/tap on the red button.

Moreover, the UI 1330 displays “Don't call! I′m okay” button (see, 1340). The user 102 may tap/click/press this button if the user 102 does not want to call the emergency contact. Also, the UI 1330 displays “Take a test again” button (see, 1342). The user 102 may press/click/tap this button if the user 102 wants to take the first test and the second test again.

In the FIG. 13C, UI 1350 of a screen that appears if symptoms of stroke are not detected in the user 102 is shown. The UI 1350 informs the user 102 with a thumbs-up icon (see, 1352) and text “No stroke symptoms detected” (see, 1354). In addition, the UI 1350 displays a “Take a test again” button (see, 1342). The user 102 may press/click/tap this button if the user 102 wants to take the first test and the second test again.

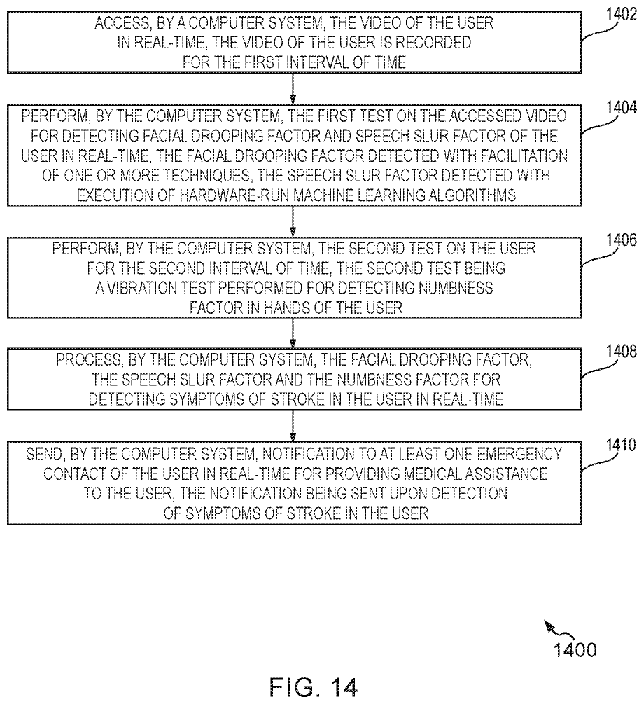

FIG. 14 is a process flow chart of a computer-implemented method 1400 for performing stroke detection, in accordance with an embodiment of the present disclosure. The method 1400 depicted in the flow chart may be executed by, for example, a computer system. The computer system is identical to the user device 104. Operations of the flow chart of method 1400, and combinations of operation in the flow chart of method 1400, may be implemented by, for example, hardware, firmware, a processor, circuitry, and/or a different device associated with the execution of software that includes one or more computer program instructions. It is noted that the operations of the method 1400 can be described and/or practiced by using a system other than these computer systems. The method 1400 starts at operation 1402.

At operation 1402, the method 1400 includes accessing, by the computer system, the video of the user in real-time. The video of the user is recorded for a first interval of time.

At operation 1404, the method 1400 includes performing, by the computer system, the first test on the accessed video for detecting the facial drooping factor and the speech slur factor of the user in real-time. The facial drooping factor is detected with facilitation of the one or more techniques. The speech slur factor is detected with execution of the machine learning algorithms.

At operation 1406, the method 1400 includes performing, by the computer system, the second test on the user for the second interval of time. The second test is the vibration test performed for detecting the numbness factor in hands of the user.

At operation 1408, the method 1400 includes processing, by the computer system, the facial drooping factor, the speech slur factor, and the numbness factor for detecting symptoms of stroke in the user in real-time.

At operation 1410, the method 1400 includes sending, by the computer system, notification to at least one emergency contact of the user in real-time for providing medical assistance to the user. The notification is sent upon detection of symptoms of stroke in the user.

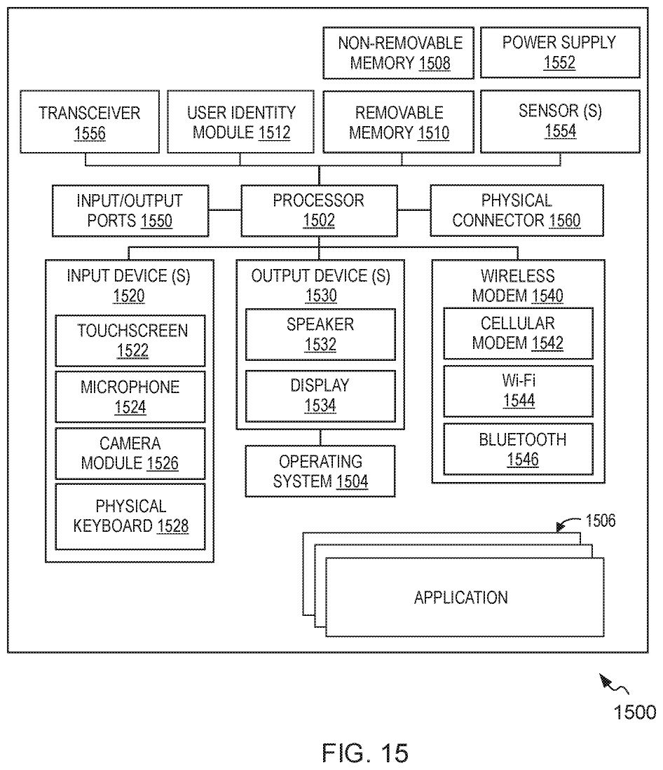

FIG. 15 is a simplified block diagram of an electronic device 1500 capable of implementing various embodiments of the present disclosure. For example, the electronic device 1500 may correspond to the user device 104 of the user 102 of FIG. 1. The electronic device 1500 is depicted to include one or more applications 1506. For example, the one or more applications 1506 may include the stroke detection application 106 of FIG. 1. The stroke detection application 106 can be an instance of the application that is hosted and managed by the server system 200. One of the one or more applications 1506 on the electronic device 1500 is capable of communicating with a server system for performing stroke detection in real-time as explained above.

It should be understood that the electronic device 1500 as illustrated and hereinafter described is merely illustrative of one type of device and should not be taken to limit the scope of the embodiments. As such, it should be appreciated that at least some of the components described below in connection with the electronic device 1500 may be optional and thus in an embodiment may include more, less, or different components than those described in connection with the embodiment of the FIG. 15. As such, among other examples, the electronic device 1500 could be any of a mobile electronic device, for example, cellular phones, tablet computers, laptops, mobile computers, personal digital assistants (PDAs), mobile televisions, mobile digital assistants, or any combination of the aforementioned, and other types of communication or multimedia devices.

The illustrated electronic device 1500 includes a controller or a processor 1502 (e.g., a signal processor, microprocessor, ASIC, or other control and processing logic circuitry) for performing such tasks as signal coding, data processing, image processing, input/output processing, power control, and/or other functions. An operating system 1504 controls the allocation and usage of the components of the electronic device 1500 and supports for one or more operations of the application (see, the applications 1506), such as the stroke detection application 106 that implements one or more of the innovative features described herein. In addition, the applications 1506 may include common mobile computing applications (e.g., email applications, calendars, contact managers, web browsers, messaging applications) or any other computing application.

The illustrated electronic device 1500 includes one or more memory components, for example, a non-removable memory 1508 and/or removable memory 1510. The non-removable memory 1508 and/or the removable memory 1510 may be collectively known as a database in an embodiment. The non-removable memory 1508 can include RAM, ROM, flash memory, a hard disk, or other well-known memory storage technologies. The removable memory 1510 can include flash memory, smart cards, or a Subscriber Identity Module (SIM). The one or more memory components can be used for storing data and/or code for running the operating system 1504 and the applications 1506. The electronic device 1500 may further include a user identity module (UIM) 1512. The UIM 1512 may be a memory device having a processor built in. The UIM 1512 may include, for example, a subscriber identity module (SIM), a universal integrated circuit card (UICC), a universal subscriber identity module (USIM), a removable user identity module (R-UIM), or any other smart card. The UIM 1512 typically stores information elements related to a mobile subscriber. The UIM 1512 in form of the SIM card is well known in Global System for Mobile (GSM) communication systems, Code Division Multiple Access (CDMA) systems, or with third-generation (3G) wireless communication protocols such as Universal Mobile Telecommunications System (UMTS), CDMA9000, wideband CDMA (WCDMA) and time division-synchronous CDMA (TD-SCDMA), or with fourth-generation (4G) wireless communication protocols such as LTE (Long-Term Evolution).

The electronic device 1500 can support one or more input devices 1520 and one or more output devices 1530. Examples of the input devices 1520 may include, but are not limited to, a touch screen/a display screen 1522 (e.g., capable of capturing finger tap inputs, finger gesture inputs, multi-finger tap inputs, multi-finger gesture inputs, or keystroke inputs from a virtual keyboard or keypad), a microphone 1524 (e.g., capable of capturing voice input), a camera module 1526 (e.g., capable of capturing still picture images and/or video images) and a physical keyboard 1528. Examples of the output devices 1530 may include, but are not limited to, a speaker 1532 and a display 1534. Other possible output devices can include piezoelectric or other haptic output devices. Some devices can serve more than one input/output function. For example, the touch screen 1522 and the display 1534 can be combined into a single input/output device.

A wireless modem 1540 can be coupled to one or more antennas (not shown in the FIG. 15) and can support two-way communications between the processor 1502 and external devices, as is well understood in the art. The wireless modem 1540 is shown generically and can include, for example, a cellular modem 1542 for communicating at long range with the mobile communication network, a Wi-Fi compatible modem 1544 for communicating at short range with an external Bluetooth-equipped device or a local wireless data network or router, and/or a Bluetooth-compatible modem 1546. The wireless modem 1540 is typically configured for communication with one or more cellular networks, such as a GSM network for data and voice communications within a single cellular network, between cellular networks, or between the electronic device 1500 and a public switched telephone network (PSTN).

The electronic device 1500 can further include one or more input/output ports 1550, a power supply 1552, one or more sensors 1554 for example, an accelerometer, a gyroscope, a compass, or an infrared proximity sensor for detecting the orientation or motion of the electronic device 1500 and biometric sensors for scanning biometric identity of an authorized user, a transceiver 1556 (for wirelessly transmitting analog or digital signals) and/or a physical connector 1560, which can be a USB port, IEEE 1294 (FireWire) port, and/or RS-232 port. The illustrated components are not required or all-inclusive, as any of the components shown can be deleted and other components can be added.

The disclosed method with reference to FIG. 14, or one or more operations of the server system 200 may be implemented using software including computer-executable instructions stored on one or more computer-readable media (e.g., non-transitory computer-readable media, such as one or more optical media discs, volatile memory components (e.g., DRAM or SRAM), or nonvolatile memory or storage components (e.g., hard drives or solid-state nonvolatile memory components, such as Flash memory components)) and executed on a computer (e.g., any suitable computer, such as a laptop computer, net book, Web book, tablet computing device, smart phone, or other mobile computing device). Such software may be executed, for example, on a single local computer or in a network environment (e.g., via the Internet, a wide-area network, a local-area network, a remote web-based server, a client-server network (such as a cloud computing network), or other such network) using one or more network computers. Additionally, any of the intermediate or final data created and used during implementation of the disclosed methods or systems may also be stored on one or more computer-readable media (e.g., non-transitory computer-readable media) and are considered to be within the scope of the disclosed technology. Furthermore, any of the software-based embodiments may be uploaded, downloaded, or remotely accessed through a suitable communication means. Such a suitable communication means includes, for example, the Internet, the World Wide Web, an intranet, software applications, cable (including fiber optic cable), magnetic communications, electromagnetic communications (including RF, microwave, and infrared communications), electronic communications, or other such communication means.

Although the invention has been described with reference to specific exemplary embodiments, it is noted that various modifications and changes may be made to these embodiments without departing from the broad spirit and scope of the invention. For example, the various operations, blocks, etc., described herein may be enabled and operated using hardware circuitry (for example, complementary metal oxide semiconductor (CMOS) based logic circuitry), firmware, software and/or any combination of hardware, firmware, and/or software (for example, embodied in a machine-readable medium). For example, the apparatuses and methods may be embodied using transistors, logic gates, and electrical circuits (for example, application specific integrated circuit (ASIC) circuitry and/or in Digital Signal Processor (DSP) circuitry).

Particularly, the server system 200 and its various components may be enabled using software and/or using transistors, logic gates, and electrical circuits (for example, integrated circuit circuitry such as ASIC circuitry). Various embodiments of the invention may include one or more computer programs stored or otherwise embodied on a computer-readable medium, wherein the computer programs are configured to cause a processor or computer to perform one or more operations. A computer-readable medium storing, embodying, or encoded with a computer program, or similar language, may be embodied as a tangible data storage device storing one or more software programs that are configured to cause a processor or computer to perform one or more operations. Such operations may be, for example, any of the steps or operations described herein. In some embodiments, the computer programs may be stored and provided to a computer using any type of non-transitory computer readable media. Non-transitory computer readable media include any type of tangible storage media. Examples of non-transitory computer readable media include magnetic storage media (such as floppy disks, magnetic tapes, hard disk drives, etc.), optical magnetic storage media (e.g., magneto-optical disks), CD-ROM (compact disc read only memory), CD-R (compact disc recordable), CD-R/W (compact disc rewritable), DVD (Digital Versatile Disc), BD (BLU-RAY® Disc), and semiconductor memories (such as mask ROM, PROM (programmable ROM), EPROM (erasable PROM), flash memory, RAM (random access memory), etc.). Additionally, a tangible data storage device may be embodied as one or more volatile memory devices, one or more non-volatile memory devices, and/or a combination of one or more volatile memory devices and non-volatile memory devices. In some embodiments, the computer programs may be provided to a computer using any type of transitory computer readable media. Examples of transitory computer readable media include electric signals, optical signals, and electromagnetic waves. Transitory computer readable media can provide the program to a computer via a wired communication line (e.g., electric wires, and optical fibers) or a wireless communication line.

Various embodiments of the disclosure, as discussed above, may be practiced with steps and/or operations in a different order, and/or with hardware elements in configurations, which are different than those which, are disclosed. Therefore, although the disclosure has been described based upon these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A computer-implemented method, comprising:
accessing, by a computer system, a video of a user in real-time, the video of the user recorded for a first interval of time;
performing, by the computer system, a first test on the accessed video for detecting a facial drooping factor and a speech slur factor of the user in real-time, the facial drooping factor detected with facilitation of one or more techniques, the speech slur factor detected with execution of machine learning algorithms;
performing, by the computer system, a second test on the user for a second interval of time, the second test being a vibration test performed for detecting a numbness factor in hands of the user;
processing, by the computer system, the facial drooping factor, the speech slur factor and the numbness factor for detecting symptoms of stroke in the user in real-time; and
sending, by the computer system, notification to at least one emergency contact of the user in real-time for providing medical assistance to the user, the notification being sent upon detection of the symptoms of the stroke in the user.

2. The computer-implemented method as claimed in claim 1, wherein the one or more techniques comprise a first technique of utilization of a machine learning model for scanning entire face of the user recorded in the accessed video for detecting the facial drooping factor in the face of the user, the machine learning model trained with sample facial data sets of non-facial muscle drooped images and facial muscle drooped images of one or more users.

3. The computer-implemented method as claimed in claim 1, wherein the one or more techniques comprise a second technique of utilization of a deep learning model for segmenting face of the user recorded in the accessed video into a plurality of facial segments in real-time, the deep learning model scanning each of the plurality of facial segments for detecting the facial drooping factor in the face of the user, the deep learning model trained using sample facial data sets of non-facial muscle drooped images and facial muscle drooped images of one or more users.

4. The computer-implemented method as claimed in claim 1, wherein the one or more techniques comprise a third technique for comparing face of the user recorded in the accessed video in real-time with face of the user already stored in a database, the comparison performed for detecting the facial drooping factor in the face of the user recorded in the accessed video in real-time.

5. The computer-implemented method as claimed in claim 1, wherein the speech slur factor is detected with facilitation of a machine learning model, the machine learning model trained with sample speech data sets of non-audio slur audio and audio slur audio of one or more users.

6. The computer-implemented method as claimed in claim 1, further comprising storing, at the computer system, a user profile of the user, wherein the user profile comprises demographic information of the user, images and videos of the user, voice samples and speech data of the user, and health information of the user, the user profile stored for personalized health reporting of the user.

7. The computer-implemented method as claimed in claim 1, further comprising:
displaying instructions on a display of the computer system for notifying the user to record the video in a camera of the computer system in real-time; and
displaying instructions on the display of the computer system for notifying the user to speak a specific phrase in the video being recorded in the camera of the computer system in real-time.

8. A computer system, comprising:
one or more sensors;
a memory comprising executable instructions; and
a processor configured to execute the instructions to cause the computer system to:
access a video of a user in real-time, the video of the user recorded for a first interval of time,
perform a first test on the accessed video to detect a facial drooping factor and a speech slur factor of the user in real-time, the facial drooping factor detected with facilitation of one or more techniques, the speech slur factor detected with execution of machine learning algorithms,
perform a second test on the user for a second interval of time, the second test being a vibration test performed to detect a numbness factor in hands of the user,
process the facial drooping factor, the speech slur factor and the numbness factor for detecting symptoms of stroke in the user in real-time, and
send notification to at least one emergency contact of the user in real-time to provide medical assistance to the user, the notification being sent upon detection of the symptoms of the stroke in the user.

9. The computer system as claimed in claim 8, wherein the one or more techniques comprise a first technique of utilization of a machine learning model to scan entire face of the user recorded in the accessed video to detect the facial drooping factor in the face of the user, the machine learning model trained with sample facial data sets of non-facial muscle drooped images and facial muscle drooped images of one or more users.

10. The computer system as claimed in claim 8, wherein the one or more techniques comprise a second technique of utilization of a deep learning model to segment face of the user recorded in the accessed video into a plurality of facial segments in real-time, the deep learning model scans each of the plurality of facial segments to detect the facial drooping factor in the face of the user, the deep learning model is trained using sample facial data sets of non-facial muscle drooped images and facial muscle drooped images of one or more users.

11. The computer system as claimed in claim 8, wherein the one or more techniques comprise a third technique to compare face of the user recorded in the accessed video in real-time with face of the user already stored in a database, the comparison performed to detect the facial drooping factor in the face of the user recorded in the accessed video in real-time.

12. The computer system as claimed in claim 8, wherein the speech slur factor is detected with facilitation of a machine learning model, the machine learning model trained with sample speech data sets of non-audio slur audio and audio slur audio of one or more users.

13. The computer system as claimed in claim 8, wherein the one or more sensors comprising at least one of: a motion detector, an accelerometer, a gyroscope, a microphone, a camera, a temperature sensor, and an ECG sensor.

14. The computer system as claimed in claim 8, wherein the computer system is further configured to store a user profile of the user, the user profile comprising demographic information of the user, images and videos of the user, voice samples and speech data of the user, and health information of the user, the user profile stored for personalized health reporting of the user.

15. The computer system as claimed in claim 8, wherein the computer system is further configured to connect with a wearable device worn by the user, the wearable device transmits additional health information of the user to the computer system in real-time.

16. A server system, comprising:
a communication interface;
a memory comprising executable instructions; and
a processing system communicably coupled to the communication interface and configured to execute the instructions to cause the server system to provide an application to a computer system, the computer system comprising one or more sensors, a memory to store the application in a machine-executable form, and a processor; the application when executed by the processor in the computer system causes the computer system to perform a method comprising:
accessing a video of a user in real-time, the video of the user recorded for a first interval of time,
performing a first test on the accessed video for detecting a facial drooping factor and a speech slur factor of the user in real-time, the facial drooping factor detected with facilitation of one or more techniques, the speech slur factor detected with execution of machine learning algorithms,
performing a second test on the user for a second interval of time, the second test being a vibration test performed for detecting a numbness factor in hands of the user,
processing the facial drooping factor, the speech slur factor and the numbness factor for detecting symptoms of stroke in the user in real-time, and
sending notification to at least one emergency contact of the user in real-time for providing medical assistance to the user, the notification being sent upon detection of the symptoms of the stroke in the user.

17. The server system as claimed in claim 16, wherein the one or more techniques comprise a first technique of utilization of a machine learning model to scan entire face of the user recorded in the accessed video to detect the facial drooping factor in the face of the user, the machine learning model trained with sample facial data sets of non-facial muscle drooped images and facial muscle drooped images of one or more users.

18. The server system as claimed in claim 16, wherein the one or more techniques comprise a second technique of utilization of a deep learning model to segment face of the user recorded in the accessed video into a plurality of facial segments in real-time, the deep learning model scans each of the plurality of facial segments to detect the facial drooping factor in the face of the user, the deep learning model is trained using sample facial data sets of non-facial muscle drooped images and facial muscle drooped images of one or more users.

19. The server system as claimed in claim 16, wherein the one or more techniques comprise a third technique to compare face of the user recorded in the accessed video in real-time with face of the user already stored in a database, the comparison performed to detect the facial drooping factor in the face of the user recorded in the accessed video in real-time.

20. The server system as claimed in claim 16, wherein the speech slur factor is detected with facilitation of a machine learning model, the machine learning model trained with sample speech data sets of non-audio slur audio and audio slur audio of one or more users.
